SEQUENCE LISTING (10 sequences)

SEQ ID NO: 1 - protein, 13 aa, Homo sapiens
DRKDQGGYTMHQD

SEQ ID NO: 2 - protein, 6 aa, Homo sapiens
TDHGAE

SEQ ID NO: 3 - protein, 7 aa, Homo sapiens
PRHLSNV

SEQ ID NO: 4 - protein, 6 aa, Homo sapiens
KTDHGA

SEQ ID NO: 5 - protein, 5 aa, Homo sapiens
AAGHV

SEQ ID NO: 6 - protein, 4 aa, Homo sapiens
EPRQ

SEQ ID NO: 7 - protein, 16 aa, Homo sapiens
TDHGAEIVYKSPVVSG

SEQ ID NO: 8 - protein, 7 aa, Homo sapiens
EFEVMED

SEQ ID NO: 9 - protein, 10 aa, Homo sapiens
GGKVQIINKK

SEQ ID NO: 10 - protein, 10 aa, Homo sapiens
DQGGYTMHQD

(19) United States
(12) Patent Application Publication
(10) Pub. No.: US 2015/0126390 A1
(43) Pub. Date: May 7, 2015

(54) MULTIPLEX AVIDITY PROFILING OF PROTEIN AGGREGATES

(71) Applicant: Washington University, St. Louis, MO (US)

(72) Inventors: Marc Diamond, St. Louis, MO (US); Scarlett Barker, St. Louis, MO (US)

(21) Appl. No.: 14/485,070

(22) Filed: Sep. 12, 2014

Related U.S. Application Data
(60) Provisional application No. 61/877,140, filed on Sep. 12, 2013.

(51) Int. Cl. G01N 33/68; G06F 19/18

(52) U.S. Cl. CPC G01N 33/6845; G01N 33/6842; G01N 33/6896; G06F 19/18; G01N 2333/4709; G01N 2800/2821; G01N 2800/2828
USPC 506/9; 702/19

(57) ABSTRACT

The present invention relates to methods for classifying conformationally-distinct aggregates of the same protein (i.e. “conformers”) by measuring minor differences in the binding avidity of a plurality of epitope-binding agents for each conformer and performing a multivariate analysis that evaluates the avidity of various antibodies for the confomers.

### CROSS REFERENCES TO RELATED APPLICATIONS

This application claims the priority of U.S. provisional application No. 61/877,140, filed Sep. 12, 2013, which is hereby incorporated by reference in its entirety.

### FIELD OF THE INVENTION

The present invention relates to methods for classifying conformationally-distinct aggregates of the same protein (i.e. “conformers”) by measuring minor differences in the binding avidity of a plurality of epitope-binding agents for each conformer and performing a multivariate analysis that evaluates the avidity of various antibodies for the conformers.

### BACKGROUND OF THE INVENTION

Emerging evidence suggests that protein aggregates underlie the pathogenesis of a variety of neurodegenerative diseases. In parallel, antibody-mediated therapies, and small molecule therapies are being brought towards the clinic. Work in the Diamond laboratory indicates that different tauopathies can now be defined at a molecular level by the underlying constellation, or “cloud” of protein aggregates that are present within the brains of individual patients. There are no facile, rapid mechanisms to define the spectrum of protein aggregates that are present in a human brain, or in peripheral tissues such as CSF and plasma. The ability to characterize the different types of aggregates present could allow a “molecular phenotype” to be defined for any individual patient, enabling refinement of specific therapies, better studies of clinical outcomes, disease progression patterns, etc.

### SUMMARY OF THE INVENTION

One aspect of the present disclosure encompasses a method for classifying a protein aggregate. The method comprises (a) contacting the protein aggregate with x number of epitope-binding agents, wherein x≧3 and none of the epitope-binding agents bind the same epitope; (b) contacting the product of step (a) with y number of a epitope-binding agents linked to a label (“labeled epitope-binding agents”) to form y types of labeled aggregate, wherein y=x and the labeled epitope-binding agents of step (b) and the epitope-binding agents from step (a) collectively recognize the same epitopes; (c) measuring the amount of label for type of labeled aggregate, wherein the amount of the label is directly proportional to binding avidity of the labeled epitope-binding agent for the protein aggregate; and (d) classifying the protein aggregate by assigning the protein aggregate to a discrete spatial location within a multivariate space having n number of axes, wherein n=y and each axis corresponds to a labeled epitope-binding agent, and wherein a coordinate along the axis is the binding avidity as measured in step (c).

In another aspect, a method for comparing the similarity of two or more protein aggregates. The method comprising (a) classifying each protein aggregate, and (b) calculating a degree of similarity between the two or more aggregates. The method of classifying each protein aggregate comprises (1) contacting the protein aggregate with x number of epitope-binding agents, wherein x≧3 and none of the epitope-binding agents bind the same epitope; (2) contacting the product of step (1) with y number of a epitope-binding agents linked to a label (“labeled epitope-binding agents”) to form y types of labeled aggregate, wherein y=x and the labeled epitope-binding agents of step (2) and the epitope-binding agents from step (1) collectively recognize the same epitopes; (3) measuring the amount of label for type of labeled aggregate, wherein the amount of the label is directly proportional to binding avidity of the labeled epitope-binding agent for the protein aggregate; and (4) classifying the protein aggregate by assigning the protein aggregate to a discrete spatial location within a multivariate space having n number of axes, wherein n=y and each axis corresponds to a labeled epitope-binding agent, and wherein a coordinate along the axis is the binding avidity as measured in step (3). The degree of similarity is calculated by determining a Euclidean distance between the spatial locations or a correlation coefficient between the binding avidities of each aggregate.

In another aspect, a method for comparing the similarity of two or more protein aggregates. The method comprising (a) classifying each protein aggregate, and (b) calculating a degree of similarity between the two or more aggregates. The degree of similarity may be calculated by determining a Euclidean distance between the spatial locations, a correlation coefficient between the binding avidities of each aggregate, or a combination thereof. The method of classifying each protein aggregate comprises (1) contacting the protein aggregate with x number of epitope-binding agents, wherein x≧3 and none of the epitope-binding agents bind the same epitope; (2) contacting the product of step (1) with y number of a epitope-binding agents linked to a label (“labeled epitope-binding agents”) to form y types of labeled aggregate, wherein y=x and the labeled epitope-binding agents of step (2) and the epitope-binding agents from step (1) collectively recognize the same epitopes; (3) measuring the amount of label for type of labeled aggregate, wherein the amount of the label is directly proportional to binding avidity of the labeled epitope-binding agent for the protein aggregate; and (4) classifying the protein aggregate by assigning the protein aggregate to a discrete spatial location within a multivariate space having n number of axes, wherein n=y and each axis corresponds to a labeled epitope-binding agent, and wherein a coordinate along the axis is the binding avidity as measured in step (3).

In another aspect, the present invention encompasses a method for assigning a location in a multivariate space to a disease associated with a protein aggregate. The method may comprise (a) obtaining a sample from a subject diagnosed with a disease associated with a protein aggregate, (b) classifying the protein aggregate, and (c) assigning the spatial location of the protein aggregate within the multivariate space to the disease of the subject. The method of classifying each protein aggregate comprises (1) contacting the protein aggregate with x number of epitope-binding agents, wherein x≧3 and none of the epitope-binding agents bind the same epitope; (2) contacting the product of step (1) with y number of a epitope-binding agents linked to a label (“labeled epitope-binding agents”) to form y types of labeled aggregate, wherein y=x and the labeled epitope-binding agents of step (2) and the epitope-binding agents from step (1) collectively recognize the same epitopes; (3) measuring the amount of label for type of labeled aggregate, wherein the amount of the label is directly proportional to binding avidity of the labeled epitope-binding agent for the protein aggregate; and (4) classifying the protein aggregate by assigning the protein aggregate to a discrete spatial location within a multivariate space having n number of axes, wherein n=y and each axis corresponds to a labeled epitope-binding agent, and wherein a coordinate along the axis is the binding avidity as measured in step (3).

Other aspects and iterations of the invention are described more thoroughly below.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts graphs showing that monomeric tau is easily discriminated from aggregated tau. (A) Microspheres coated with a monoclonal antibody (HJ 9.3) were incubated with recombinant tau in either fibrillar or monomeric form. These complexes were then incubated with the same monoclonal antibody labeled with a fluorescent dye. An increase in the median fluorescence intensity indicates an increase in the number of antibodies bound to each microsphere. Using the same monoclonal antibody for capture and detection prevents monomeric tau from increasing the fluorescence signal, as each monomer contains only one epitope for any monoclonal antibody. The specificity of this method is exemplified by comparing PBS and BSA to monomeric tau purified from the brain homogenate of a patient diagnosed with Alzheimer's disease (B). The signal that arises from monomeric human tau is not significantly different from background, ensuring that an increase in fluorescence can be attributed to the aggregated material in the sample.

FIG. 2 depicts a graph showing that multiplexing shows structural differences between the dominant tau species in AD and PSP brains. Brain homogenates from the middle frontal gyrus of eight tauopathy patients were characterized by the microsphere-based sandwich system using three distinct monoclonal antibodies. Four patients had a neuropathological diagnosis of Progressive Supranuclear Palsy (PSP) while four others had a neuropathological diagnosis of Alzheimer's disease (AD). The brain homogenates were normalized by total protein content (as determined by a BCA assay) and added to the microspheres in excess. The brain homogenate from a tau-knockout mouse was used as a negative control for nonspecific binding. One antibody (9.3) showed similar levels of binding across all brains, suggesting that differences are not due to vastly different concentrations of tau. Interestingly, the other antibodies displayed gradations of binding with a clear distinction between the AD and PSP homogenates. K means analysis was used to group the homogenates into two families based on binding across all three antibodies and was 100% disease specific. In this case, only one antibody (8.1) is needed to separate the homogenates by disease. These findings suggest that the dominant tau species in AD and PSP are structurally distinct, displaying different epitope availabilities for the binding of 8.1 and 8.5.

FIG. 3 depicts a diagram showing clustering analyses based on binding intensities to four monoclonal antibodies. Brain homogenates from 21 tauopathy patients were characterized by the microsphere-based sandwich system using four monoclonal antibodies. Clustering analyses grouped all of the AD patients in one family and the two Corticobasal degeneration (CBD) patients in a distinct family. PSP patients were grouped in the same principal family, but one homogenate (3) was deemed somewhat distinct. Other tauopathies showed more heterogeneity. These findings suggest that certain tauopathies may be more uniform than others in terms of tau conformations present. Current work is being done to correlate structural outliers to clinical outliers (e.g. was there something clinically atypical about patient 3 or patient 12 that may account for their separation from other patients of the same diagnoses?).

FIG. 4 depicts a diagram showing that the same clustering is achieved using a non-overlapping set of monoclonal antibodies. Brain homogenates from 21 tauopathy patients were characterized by the microsphere-based sandwich system using three monoclonal antibodies, none of which were used in the previous analysis. Interestingly, the same families arise. AD patients are grouped together uniformly. CBD patients are also grouped together, but in a distinct family. Patient 3 is once again deemed an outlier PSP patient. Patient 12 is deemed an outlier AGD patient, and is once again grouped with the CBD family. These findings exemplify the power of structure mapping by multiplexing; the dimensionality achieved using antibodies that bind to distinct regions of tau is sufficient to group tauopathy patients into families without relying on a “special” antibody to discriminate conformations.

FIG. 5 depicts graphs showing aggregated tau is discriminated from monomeric tau. (A) Two monoclonal cell lines containing equivalent amounts of tau RD fused to YFP were lysed and incubated with a monoclonal anti-tau antibody conjugated to a fluorescent dye. One cell line contained only diffuse tau RD-YFP (red) while the other stably propagated tau aggregates (blue). The solution was diluted and passed through a flow cytometer such that each event detected would provide a measure of both YFP and antibody fluorescence. The monomeric cell line displayed one YFP peak on the lower end of the fluorescence spectrum, indicating homogeneity in size. The cell line containing aggregated tau, however, displayed a range of YFP fluorescence, indicating a larger range of sizes. (B) Plotting YFP fluorescence against antibody fluorescence shows one discrete level of antibody binding in the monomeric cell line, as expected. The cell line containing aggregated tau shows a logarithmic relationship between YFP fluorescence and antibody fluorescence, indicating that more antibodies bind to larger tau aggregates. Importantly, the relationship between antibody fluorescence and YFP fluorescence is consistent throughout the population, suggesting the presence of a single conformation of tau. This has been confirmed both biochemically and morphologically.

FIG. 6 depicts a graph showing that antibody binding is protein specific. To test for the possibility of system artifact, aggregated tau derived from the monoclonal cell line described previously was incubated with either an anti-tau antibody or an anti-Aβ antibody, both conjugated to a fluorescent dye. As expected, the anti-tau antibody displayed greater binding to increasing aggregate sizes, while the anti-Aβ antibody did not. This data suggests that dual fluorescence positivity is not a result of coincidence, but of true antibody-antigen binding. Background positivity can be reduced approximately threefold by conjugating the same antibody to two distinct fluorescent dyes and gating for dual positivity among antibody fluorescence.

FIG. 7 depicts a graph showing that distinct conformations of tau are identified. Two monoclonal cell lines propagating distinct conformations of tau (as determined by morphological and biochemical assays) were lysed and incubated with a monoclonal antibody conjugated to a fluorescent tag. The strains show differential antibody binding per unit of tau, suggesting that there are fewer epitopes spatially available in one conformation. By itself, this data can conclude that the conformers in each cell line are structurally different.

FIG. 8 depicts graphs showing that multiple strains within the same sample can be discriminated. (A,B) The two strains described previously (9 and 10) were incubated in the same sample along with a monoclonal antibody that shows differential binding patterns to each strain. (A) Depicts analysis by flow cytometry, providing a measure of both YFP and antibody fluorescence. (B) The ratio of antibody fluorescence per unit of tau was calculated for every event and plotted in a frequency histogram. Each of the two peaks in the histogram corresponds to an individual strain.

FIG. 9 depicts a graph showing fingerprinting cell-derived tau strains. Brain homogenates from tauopathy patients were applied to cells producing tau RD-YFP in order to “seed” the diffuse tau and create stable cell lines able to propagate tau inclusions of various morphologies. Morphologically, two cell lines derived from a Pick's disease (i.e. PiD #1 and PiD #2) brain appeared to propagate the same strain. Likewise, two cell lines derived from an Alzheimer's disease brain (i.e. AD #1 and AD #2) appeared to propagate the same strain, which was morphologically distinct from the Pick's strains. These cell lines were lysed and incubated with three monoclonal antibodies. As shown, the Pick's strains display the same pattern of antibody binding and the Alzheimer's strains display the same pattern of antibody binding. These visual similarities have been confirmed with Matlab. This method is able to replicate the cellular data and group tau conformations in a more efficient and quantitative manner.

FIG. 10 depicts a graph showing the application of the fingerprinting method to human samples. Immunoprecipitated tau from two human brains (D22 and D6) was incubated with a polyclonal anti-tau antibody and a monoclonal anti-tau antibody. The polyclonal antibody serves as a proxy for aggregate size. These samples display positive binding to the monoclonal antibody compared to Htt fibrils, used a negative control. Additionally, the samples display differential binding to the antibody, suggesting the presence of different tau strains. Once this work is extended it has the potential to identify multiple strains within a sample and group samples based on binding similarities.

FIG. 11 depicts graphs and a Western blot showing the preparation of three distinct fibril structures. Full-length recombinant tau (2N, 4R) was fibrillized under three conditions. (A) After 120 hours of undisrupted fibrillization the pellet fractions of all preparations showed significantly more binding to Thioflavin T than did monomeric tau. (B) Limited proteolysis with pronase revealed distinct protease resistant bands between fibrils types: Preparation A displayed a single band smaller than 10 kDa; B displayed a doublet approximately 10 kDa in size; C displayed a doublet greater than 10 kDa (C) Far-UV circular dichroism revealed absorption differences between the heparin fibrils (preparations A and B) and those created with ODS (preparation C). Both monomeric tau and preparation C featured ellipticity minima between 200 and 205 nm, consistent with random coil predominating the structure. Preparations A and B exhibited ellipticity minima at approximately 220 nm, consistent with predominant beta sheet structures. (D) Titrating fibril preparations onto a biosensor cell line triggered differing degrees of intracellular tau aggregation, as detected by FRET flow cytometry. Preparation A had the highest seeding efficiency, B was intermediate, and C was the least potent (see also Table 2).

FIG. 12 illustrates embodiments of the invention where the protein aggregate is amyloid and epitope-binding agents are antibodies. (A,B) This illustration is not limiting. The antibodies depicted may be substituted with any epitope-biding agent. Panel (A) illustrates the concept of a “sandwich assay”. By requiring that the first and the second epitope-binding agent detect the same epitope, the assay selectively measures multimer binding. In this embodiment, microspheres are coated with a single monoclonal antibody, with an identical monoclonal antibody used for secondary detection. If a monomer is trapped, its epitope is occupied, and the detection antibody doesn't bind (left side of the panel). If a multimer is trapped, multiple epitopes are available and the secondary antibody binds to produce a signal (right side of panel). Panel (B) illustrates the concept of multiplex avidity profiling (MAP). Monoclonal antibodies that bind distinct epitopes (illustrated by three different colors) on a given protein are selected. When the protein assumes amyloid structures of different conformation, the avidity of a given antibody for its epitope may vary. Through use of the sandwich assay described in (A), it is possible to measure the relative avidity of an antibody for different structures, and thus to use multiple antibodies in parallel analyses (i.e. a single antibody per sample, not depicted) or in combination (depicted) to derive a profile of relative avidities. MAP thus allows characterization of multiple amyloid structures, without the need for conformation-specific antibodies.

FIG. 13 depicts illustrations and graphs showing MAP identifies three distinct fibril conformations. (A) Five monoclonal antibodies, with epitopes spanning the length of the tau protein, were used to generate MAPs. After linking each monoclonal to a microsphere, samples were incubated with the fibrils in technical triplicates, followed by exposure to the same monoclonal antibody for detection. Binding intensities were recorded using flow cytometry. (B) The three fibril preparations each exhibited distinct patterns of antibody binding. Each MAP is defined by a position in five-dimensional space in which individual axes represent the signals from individual antibodies. Two independent measures were calculated: (C) the Euclidean distance between each pairwise sample combination in 5-dimensional space, and (D) the correlation coefficient between each pairwise sample combination. The color-coding indicates the degree of similarity for each pairwise comparison, with blue corresponding to samples that are most similar and red corresponding to samples that are least similar. Both measures indicate high similarity between technical replicates within a fibril preparation, which are much more similar than different fibril preparations. (E) K means clustering based on Euclidean distance grouped the samples by fibril preparation at K=3.

FIG. 14 depicts graphs and a diagram showing that MAP accurately groups tauopathies by syndrome. (A-C) MAPs were generated for all human brain samples, using technical triplicates, and experimental quadruplicates. Data points were then compared by Euclidean distance and correlation coefficient, using color coding to compare relatedness of samples. (A) Euclidean distance matrix shows a higher degree of similarity between samples of an individual tauopathy than across tauopathies. (B) The correlation coefficient matrix also shows a high degree of similarity between samples of an individual tauopathy, but poor separation between the AD and CBD patients. (C) K means clustering based on Euclidean distance accurately binned samples by tauopathy at K=3. At K=4 the PSP patients consistently separate into two subgroups. Clusters generated at K≧5 are not consistent across experiments, indicating that only 4 groups can be validated.

FIG. 15 depicts a graph showing the efficiency of fibrillization. To generate conformationally distinct aggregate populations, recombinant, full-length (2N,4R) monomeric tau was fibrillized under the three conditions (Table 1): A, with heparin (8 μM) at 37° C.; B, with heparin (8 μM) at 22° C.; C with octadecyl sulfate (50 μM) at 37° C. After 120 h, over 88% of the tau in each preparation was insoluble, indicating that all reactions proceeded to near completion.

FIG. 16 depicts graphs showing a bead-based sandwich assay detects multimers from tauopathy brain. (A) A bead-based sandwich assay was used with four different anti-tau monoclonal antibodies to detect tau aggregates in brain lysate. After binding to antibody-coated microspheres, and incubation with the same fluorescently tagged antibodies, the fluorescence of each individual microsphere was measured by flow cytometry. Brain lysate from a tau knockout mouse was used as a negative control. Alzheimer disease (AD) brain lysate with confirmed tau pathology had signal with all antibody pairs, but not Huntington disease (HD) brain lysate without detectable tau pathology. (B) Titration of AD brain homogenate showed increasing signal, but not the HD brain lysate.

FIG. 17 depicts graphs and diagrams showing individual metrics cannot cluster samples by syndrome. (A-F) Brain samples from 17 patients were analyzed. (A) We determined the seeding activity present in brain lysates using a cellular biosensor assay. Seeding activity varied across samples. (B) Using the results from (D) and (E) the ratio of soluble/insoluble tau was calculated across the samples. (C) We combined the above analyses with age at death for K-means clustering. None of the above measures accurately grouped samples by syndrome. By contrast MAP clustered the samples accurately. (D) Homogenized brain samples were ultracentrifuged and then separated into pellet and supernatant fractions. Using an immunoblot and FIJI software for quantification, these samples were compared to known quantities of recombinant tau in order to determine the relative concentrations of tau in the supernatant and pellet fractions. (E, F) Standard curves were generated of tau in the supernatant and tau in the pellet.

FIG. 18 depicts a diagram showing that individual antibodies do not reliably predict syndrome. K means clustering applied to the binding signals of individual antibodies does not reliably group samples by tauopathy with complete specificity at K=3. Thus, multiplexing adds power to the clustering analysis.

FIG. 19 depicts graphs showing that MAP by correlation coefficient is unaffected by aggregate load. (A,B) To test the relative accuracy of Euclidean distance vs. correlation coefficient in the setting of variable tau aggregate load, we spiked a single AD tauopathy brain lysate into an HD brain lysate (which has no tau aggregation) at various dilutions. We applied MAP to determine the effect of dilution on the signal, using three anti-tau monoclonal antibodies. (A) All antibody signals increased with increasing concentrations of AD brain lysate, thus affecting the position of each sample in three-dimensional space. (B) Despite the change in aggregate load, the correlation coefficients between samples remained consistently high.

### DETAILED DESCRIPTION OF THE INVENTION

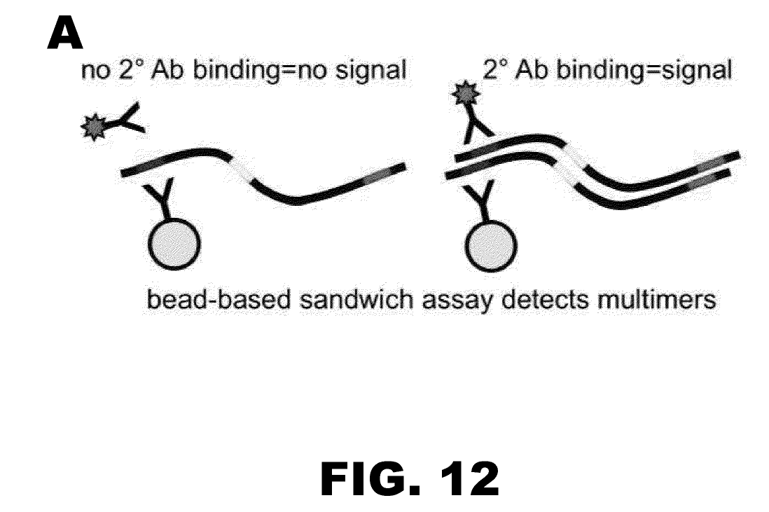

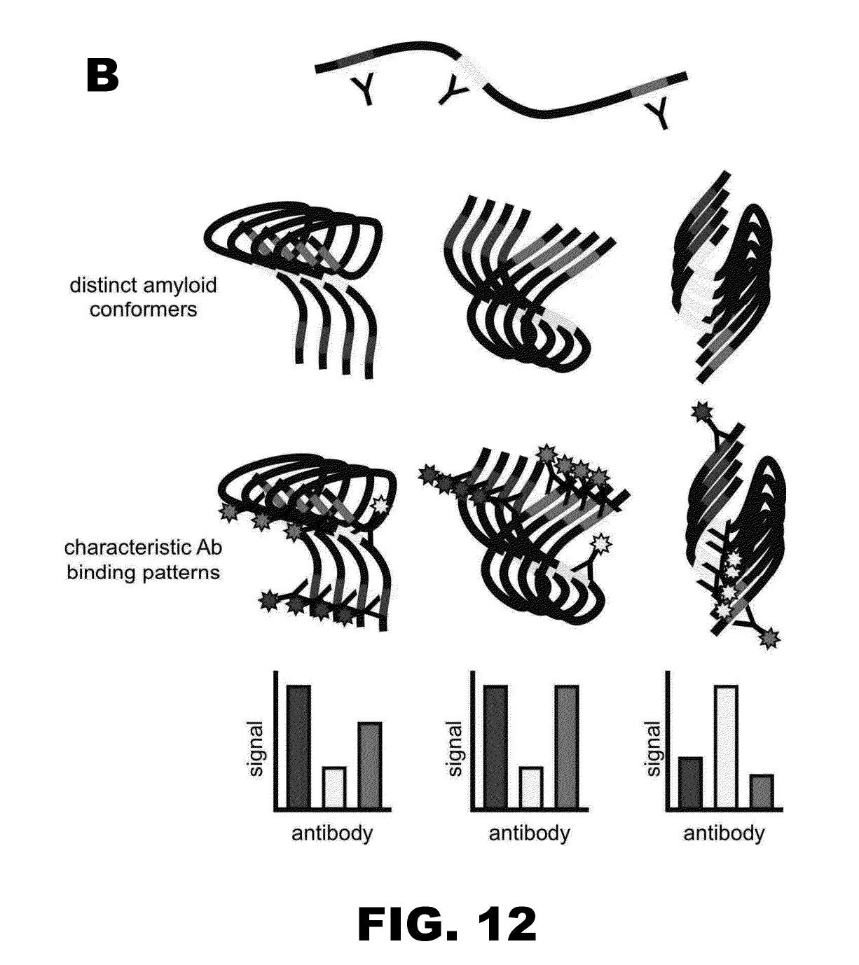

Applicants have discovered that it is possible to discriminate between conformationally-distinct aggregates of the same protein (i.e. “conformers”) by measuring minor differences in the binding avidity of a plurality of epitope-binding agents for each conformer and performing a multivariate analysis that evaluates the avidity of various antibodies for the confomers. An aspect of the invention is illustrated in FIG. 12B, in which an epitope-binding agent is exemplified as an antibody. As shown in the illustration, the availability of a group of epitopes may vary between conformers. Therefore, an epitope-binding agent may recognize its cognate epitope with slightly different avidity depending upon the structure of the protein aggregate. In another aspect of the invention, a protein aggregate may be assigned to a discrete spatial location within a multivariate space having n number of axis, wherein each axis within the multivariate space corresponds to an epitope-binding agent (i.e. n equals the number of epitope-binding agents) and the coordinate along the axis is the binding avidity. For any two aggregates that differ in the relative degree of availability of x number of epitopes, as measured by binding avidity for y number of epitope-binding agents (where x=y), the aggregates will occupy discrete locations within the multivariate space. Another aspect of the invention, then, provides means to discriminate between two or more conformers. Advantageously, the invention provides means for facile discrimination of conformers without relying solely on conformation-specific antibodies, or other less-quantitative methods such as limited proteolysis.

Another aspect of the invention encompasses methods to classify the structural conformation(s) of a protein aggregate associated with a particular disease. Accordingly, the invention also encompasses methods to assign the spatial location of a protein aggregate within the multivariate space to a disease when the protein aggregate is obtained from a subject with the disease.

Other aspects of the invention are described in further detail below.

### I. Protein Aggregate

The term “protein aggregate” refers to an accumulation of two or more misfolded proteins. A protein aggregate may be comprised of recombinant protein, naturally occurring protein, or a combination thereof. The term “protein”, as used herein, includes peptides, polypeptides, fusion proteins, naturally occurring proteins, and recombinant or artificially synthesized proteins, as well as analogs, fragments, derivatives or combinations thereof. As used herein, “recombinant protein” refers to a protein that is encoded by a nucleic acid sequence that is not typically present in the wild-type genome of the cell expressing it. Methods of making and expressing recombinant protein are well known in the art. As used herein, “naturally occurring protein” refers to a protein that is encoded by a nucleic acid sequence that is typically present in the wild-type genome of the cell expressing it. A protein aggregate may or may not be associated with a disease or disease pathology.

In an aspect, a protein aggregate may be comprised of any protein with an aggregation-prone domain. The term “aggregation-prone domain” refers to a region of the amino acid sequence of a protein that promotes the protein's aggregation. For example, the tau protein has either three or four repeat regions that constitute the aggregation-prone core of the protein, which is often termed the repeat domain (RD). Expression of the tau RD causes pathology in transgenic mice, and it reliably forms fibrils in cultured cells. As another example, the androgen receptor (AR) and huntingtin (htt) have expanded tracts of glutamines that contribute to formation of perinuclear and nuclear aggregates of these proteins. In some embodiments, an aggregation-prone domain is unique to a single protein. In other embodiments, an aggregation prone domain may be common to more than one protein. Aggregation-prone domains are well known in the art, or may be predicted through computational modeling.

In another aspect, a protein aggregate may be comprised of a pathological protein. The term “pathological protein”, as used herein, refers to a protein that aggregates, whereby aggregation is closely linked to disease pathology. Pathological proteins are well-known in the art. A pathological protein may be a polyglutamine expansion protein or a non-polyglutamine expansion protein.

Polyglutamine expansion diseases are a class of neurodegenerative diseases associated with pathological aggregation of a protein containing expanded tracts of glutamines (e.g. a polyglutamine expansion protein). Pathological polyglutamine expansion proteins (and their related disorders) may include, but are not limited to, htt (Huntington's disease), androgen receptor (AR; spinobulbar muscular atrophy), ATN1 (dentatorubropallidoluysian atrophy), ATXN1 (Spinocerebellar ataxia Type 1), ATXN2, (Spinocerebellar ataxia Type 2), ATXN3, (Spinocerebellar ataxia Type 3), CACNA1A (Spinocerebellar ataxia Type 6), ATXN7 (Spinocerebellar ataxia Type 7), and TBP (Spinocerebellar ataxia Type 17).

Non-limiting examples of non-polyglutamine expansion proteins include tau, synuclein, superoxide dismutase (SOD1), PABPN1, amyloid beta peptide, serpin, transthyretin, TDP-43 (TARDBP), valosin containing peptide (VCP), hnRNPA2B1 and hnRNPA1 and prion protein.

Tauopathies are class of neurodegenerative diseases associated with the pathological aggregation of tau protein into fibrillar tau aggregates. Exemplary disorders that have clinical signs or symptoms associated with tau aggregation include, but are not limited to, progressive supranuclear palsy, dementia pugilistica (chronic traumatic encephalopathy), frontotemporal dementia and parkinsonism linked to chromosome 17, Lytico-Bodig disease (Parkinson-dementia complex of Guam), tangle-predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, argyrophilic grain disease (AGD), Frontotemporal lobar degeneration, Alzheimer's Disease, and frontotemporal dementia.

Exemplary diseases that have symptoms associated with SOD1 aggregation may include amyotrophic lateral sclerosis (Lou Gehrig's disease). Exemplary disorders that have symptoms associated with PABPN1 aggregation may include oculopharyngeal muscular dystrophy.

Exemplary diseases that have symptoms associated with synuclein aggregation may include Parkinson's disease, Alzheimer's disease, Lewy body disease and other neurodegenerative diseases.

Exemplary diseases that have symptoms associated with serpin aggregation (“serpinopathies”) may include alpha 1-antitrypsin deficiency which may cause familial emphysema and liver cirrhosis, certain familial forms of thrombosis related to antithrombin deficiency, types 1 and 2 hereditary angioedema related to deficiency of C1-inhibitor, and familial encephalopathy with neuroserpin inclusion bodies.

Exemplary diseases that have symptoms associated with transthyretin aggregation may include senile systemic amyloidosis, familial amyloid polyneuropathy, and familial amyloid cardiomyopathy.

Exemplary diseases that have symptoms associated with prion aggregation may include scrapie, bovine spongiform encephalopathy (mad cow disease), transmissible mink encephalopathy, chronic wasting disease, feline spongiform encephalopathy, exotic ungulate encephalopathy, Creutzfeldt-Jakob diseases, Gerstmann-Straussler-Scheinker syndrome, fatal familial insomnia, and Kuru.

Exemplary diseases that have symptoms associated with TDP-43 aggregation may include FTLD-TDP and chronic traumatic encephalopathy.

Exemplary diseases that have symptoms associated with amyloid beta peptide aggregation may include Alzheimer's disease, Lewy body disease, cerebral amyloid angiopathy, inclusion body myositis and traumatic brain injury.

Exemplary diseases that are associated with VCP aggregation include Inclusion body myopathy with early-onset Paget disease and frontotemporal dementia (IBMPFD).

Exemplary diseases caused by hnRNPA2B1 and hnRNPA1 include multisystem proteinopathy and ALS.

In another aspect, a protein aggregate may or may not have an ordered structure. In preferred embodiments of the invention, a protein aggregate is an amyloid. An amyloid is a paracrystalline, ordered protein assembly. An amyloid generally has a cross-beta structure, in vivo or in vitro. Most, but not all, cross-beta structures may be identified by apple-green birefringence when stained with Congo Red and seen under polarized light, or by X-ray fiber diffraction patterns. Amyloid may be located in the periphery or in the central nervous system, or both. Amyloids are well known in the art. See, for example, Eisenberg et al. Cell. 2012 Mar. 16; 148(6):1188-203.

For amyloid to form, a nucleus must template the bonding pattern of the fiber spine. As used herein, the term “amyloid seed” refers to an amyloid that is capable of nucleating or “seeding” further amyloid protein aggregation in vitro or in vivo. Accordingly, the term “amyloid” includes “amyloid seed’.

An amyloid may or may not be disease associated. An amyloid may also be associated with more than one disease. Amyloids associated with a disease (and their related disease) may include, but are not limited to, aggregates comprised of amyloid beta peptide (Aβ, Alzheimer's disease, cerebral amyloid angiopathy), IAPP (amylin, AlAPP, Diabetes mellitus type 2), alpha-synuclein (Parkinson's disease), PrP^{Sc} (APrP, transmissible spongiform encephalopathy and fatal familial insomnia), huntingtin (Huntington's disease), calcitonin (ACal, medullary carcinoma of the thyroid), atrial natriuretic factor (AANF, cardiac arrhythmia, isolated atrial amyloidosis), apolipoprotein A1 (AApoA1, atherosclerosis, Alzheimer's Disease), serum amyloid A (AA, rheumatoid arthritis), medin (AMed, aortic medial amyloid), prolactin (APro, prolactinomas), transthyretin (ATTR, familial amyloid polyneuropathy), lysozyme (ALys, hereditary non-neuropathic systemic amyloidosis), Beta 2 microglobulin (Aβ2M, dialysis related amyloidosis), gelsolin (AGel, Finnish amyloidosis), keratoepithelin (AKer, lattice corneal dystrophy), cystatin (ACys, cerebral amyloid angiopathy (Icelandic type)), immunoglobulin light chain AL (AL, systemic AL amyloidosis), S-IBM (sporadic inclusion body myositis), and tau (tauopathies). Amyloids not specifically associated with a disorder include, but are not limited to, native amyloids in organisms, peptide/protein hormones stored as amyloids within endocrine secretory granules, proteins and peptides engineered to make amyloid display specific properties such as ligands that target cell surface receptors, several yeast prions (e.g. PSI+, Sup35p, URE3, Ure2p, PIN+, Rnq1p, SWI1+, Swi1p, OCT8+, Cyc8p), and functional amyloids in environmental biofilms. Native amyloids in an organism include, but are not limited to, curli fibrils produced by E. coli, Salmonella, other members of the Enterobacteriales, and other phyla containing the Csg operon, functional amyloids in Pseuodomonas encoded by the Fap operon, chaplins from Streptomyces coelicolor, Podospora Anserina Prion Het-s, Malarial coat protein, Spider silk, mammalian melanosomes (pMel), tissue-type plasminogen activator (tPA), ApCPEB protein and its homologis with a glutamine-rich domain, and Pmel17 derived amyloid within the melansomal matrix.

In another aspect, a protein aggregate may be in a biological sample. Suitable biological samples include tissue samples or bodily fluids. The choice of the sample will depend, in part, upon the protein aggregate. For example, if a protein aggregate is typically found in the central nervous system or is associated with a neurodegenerative disease, non-limiting examples of suitable tissue may be include brain tissue, spinal cord tissue and central nervous system (CNS) microvascular tissue, and non-limiting examples of suitable biological fluids include cerebral spinal fluid, interstitial fluid, blood, serum or plasma. Alternatively, if a protein aggregate is typically found in the periphery or is associated with a peripheral disease, non-limiting examples of suitable tissue may include blood, serum, plasma, saliva, sputum, lymph or urine. Tissue samples may be processed into a homogenate, a cell extract, a membranous fraction, or a protein extract. Biological fluids may be used “as is”, the cellular components may be isolated from the fluid, or a protein faction may be isolated from the fluid using standard techniques. The method of collecting a sample can and will vary depending upon the nature of the sample. Methods for collecting tissue and biological fluid samples are well known in the art. Generally speaking, the method preferably maintains the integrity of the sample such that conformation of the protein aggregate can be accurately analyzed according to the invention. Further processing of a biological sample may be necessary after collection of the biological sample but before using a biological sample, as well as a fraction or components obtained therefrom, in a method of the invention. For example, a biological sample may be concentrated in order to provide a greater amount of the protein aggregate, or protein aggregate may be partially or completely purified from other components of a biological sample. Suitable methods to separate a protein aggregate from other components of a biological sample include, but are not limited to, chromatography, immunoprecipitation, affinity purification, and adsorption. As a non-limiting example, enrichment of protein amyloid “seeds” via affinity chromatography, bead-based purification, and/or other immuno-affinity purification methods may be performed using anti-protein antibodies (e.g. anti-tau) or anti-amyloid antibodies. As an alternative, or in addition to, enrichment of protein amyloid “seeds” may be achieved using amyloid-binding aptamers, short polypeptides, or small molecules to enrich seeds.

Biological samples may be obtained from any suitable subject. Typically, the subject is diagnosed with a disease associated with pathological protein aggregation, preferably of an amyloid protein, or a subject with clinical signs or symptoms of a disease associated with the pathological protein aggregation, preferably of an amyloid protein. Suitable subjects may include a human, a livestock animal, a companion animal, a lab animal, or a zoological animal. In one embodiment, a subject may be a rodent, e.g. a mouse, a rat, a guinea pig, etc. In another embodiment, a subject may be a livestock animal. Non-limiting examples of suitable livestock animals may include pigs, cows, horses, goats, sheep, llamas and alpacas. In yet another embodiment, a subject may be a companion animal. Non-limiting examples of companion animals may include pets such as dogs, cats, rabbits, and birds. In yet another embodiment, a subject may be a zoological animal. As used herein, a “zoological animal” refers to an animal that may be found in a zoo. Such animals may include non-human primates, large cats, wolves, and bears. In a preferred embodiment, a subject is human.

In a specific embodiment, a protein aggregate may be comprised of pathological protein associated with a neurodegenerative disease. In another specific embodiment, a protein aggregate may be comprised of protein selected from the group consisting of prion protein, tau protein, alpha-synuclein protein, amyloid beta peptide, TDP-43, and htt. In another specific embodiment, a protein aggregate may be an amyloid. In another specific embodiment, a protein aggregate may be comprised a protein selected from the group consisting of amyloid beta peptide, PrP^{sc}, huntingtin, calcitonin, apolipoprotein A1, transthyretin, tau, and cystatin. In another specific embodiment, a protein aggregate may be comprised a protein selected from the group consisting of atrial natriuretic factor, apolipoprotein A1, serum amyloid, medin, prolactin, lysozyme, Beta 2 microglobulin, gelsolin, keratoepithelin, immunoglobulin light chain AL, and S-IBM.

### II. Epitope-Binding Agent

The term “epitope-binding agent” refers to an antibody, an aptamer, a nucleic acid, an oligonucleic acid, an amino acid, a peptide, a polypeptide, a protein, a lipid, a metabolite, a small molecule, or a fragment thereof that recognizes and is capable of binding to an epitope exposed on the surface of a protein aggregate. The epitope may be a linear epitope or may be a conformational epitope. In some embodiments, an epitope is a linear epitope and the epitope-binding agent is an antibody or an aptamer. As used herein, the term “linear epitope” refers to an epitope consisting of a linear (or continuous) sequence of amino acids. In other embodiments, an epitope is a conformational epitope and the epitope-binding agent is an antibody or an aptamer. As used herein, the term “conformational epitope” refers to an epitope consisting of discontinuous amino acids on the surface of a protein aggregate that have a specific three-dimensional shape.

Methods of generating an epitope-binding agent to a protein aggregate are well known in the art. For example, monoclonal antibodies may be generated using a suitable hybridoma as would be readily understood by those of ordinary skill in the art. In the preferred process, a protein in accordance with the invention is first identified and isolated. Next, the protein is isolated and/or purified in any of a number of suitable ways commonly known in the art, or after the protein is sequenced, the protein used in the monoclonal process may be produced by recombinant means as would be commonly used in the art and then purified for use. In one suitable process, monoclonal antibodies may be generated from proteins isolated and purified as described above by mixing the protein with an adjuvant, and injecting the mixture into a laboratory animal. Immunization protocols may consist of a first injection (using complete Freund's adjuvant), two subsequent booster injections (with incomplete Freund's adjuvant) at three-week intervals, and one final booster injection without adjuvant three days prior to fusion. For hybridoma production, the laboratory animal may be sacrificed and their spleen removed aseptically. Antibody secreting cells may be isolated and mixed with myeloma cells (NS1) using drop-wise addition of polyethylene glycol. After the fusion, cells may be diluted in selective medium (vitamin-supplemented DMEM/HAT) and plated at low densities in multiwell tissue culture dishes. Tissue supernatants from the resulting fusion may be screened by both ELISA and immunoblot techniques. Cells from these positive wells may be grown and single cell cloned by limiting dilution, and supernatants subjected to one more round of screening by both ELISA and immunoblot. Positive clones may be identified, and monoclonal antibodies collected as hybridoma supernatants. For example, Yamada et al., J of Neuroscience 2011; 31(37):13110-13117, hereby incorporated by reference in its entirety, discloses a method of generating antibodies to tau protein. Nucleic acid aptamers may be generated through repeated rounds of in vitro selection or equivalently, SELEX (systematic evolution of ligands by exponential enrichment) to bind to a protein in accordance with the invention. Peptide aptamers may be generated from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers.

In preferred embodiments, an epitope-binding agent binds to an epitope exposed on the surface of two or more conformers. A person of ordinary skill in the art can experimentally determine if an epitope-binding agent binds to an epitope exposed on the surface of two more conformers by methods well known in the art. For example, epitope mapping may be used to identify binding sites of an epitope-binding agent. Methods for epitope mapping include, but are not limited to, x-ray co-crystallography, array-based oligo-peptide scanning (overlapping peptide scan or pepscan analysis), site-directed mutagenesis, mutagenesis mapping, phage display and limited proteolysis. Epitope mapping may be used to identify linear epitopes or conformational epitopes.

In some embodiments, a protein aggregate is a tau aggregate and the epitope-binding agent binds to an epitope on the tau aggregate. Epitopes on tau aggregates and epitope-binding agents that bind thereto are known in the art. In a specific embodiment, a protein aggregate is a tau aggregate and the epitope-binding agent binds to an epitope on the tau aggregate selected from the group consisting of DRKDQGGYTMHQD (SEQ ID NO:1), TDHGAE (SEQ ID NO:2), PRHLSNV (SEQ ID NO:3), KTDHGA (SEQ ID NO:4), AAGHV (SEQ ID NO:5), EPRQ (SEQ ID NO:6), TDHGAEIVYKSPVVSG (SEQ ID NO:7), EFEVMED (SEQ ID NO:8), GGKVQIINKK (SEQ ID NO:9) and DQGGYTMHQD (SEQ ID NO:10). In still other embodiments, a protein aggregate is a tau aggregate and the epitope-binding agent binds to an epitope on the tau aggregate selected from the group consisting of PRHLSNV (SEQ ID NO:3), AAGHV (SEQ ID NO:5), GGKVQIINKK (SEQ ID NO:9) and DRKDQGGYTMHQD (SEQ ID NO:1). Suitable epitope-binding agents include, but are not limited to, HJ 8.1, HJ 8.1.1, HJ 8.1.2, HJ 8.2, HJ 8.3, HJ 8.4, HJ 8.5, HJ 8.7, HJ 8.8, HJ 9.1, HJ 9.2, HJ 9.3, HJ 9.4, and HJ 9.5. In an exemplary embodiment, the epitope-binding agent is selected from the group consisting of HJ 8.1, HJ 8.2, HJ 8.5, HJ 8.7, and HJ 9.3. In a specific embodiment, the epitope-binding agent is selected from the group consisting of HJ 8.1, HJ 8.2, HJ 8.7 and HJ 9.3 Other suitable antibodies are known in the art. For example, suitable tau antibodies include, but are not limited to, antibodies described in PCT/US2013/049333, incorporated herein in its entirety by reference.

In other embodiments, a protein aggregate is comprised of amyloid beta peptide and epitope-binding agent binds to an epitope on the amyloid beta peptide aggregate. Epitopes on amyloid beta peptide aggregates and epitope-binding agents that bind thereto are known in the art. Non-limiting examples of epitope-binding agents that bind amyloid beta peptide aggregate include MOAB-2, MABN638, MABN640, MABN637, 05-831-I, MABN254, MAB8768, AB2500, AB5078P, AB5737, AB2539, and B-4.

In other embodiments, a protein aggregate is a prion aggregate and epitope-binding agent binds to an epitope on the prion aggregate. Epitopes on prion aggregates and epitope-binding agents that bind thereto are known in the art. Non-limiting examples of epitope-binding agents that bind prion aggregate include SAF 84, AB6664, MAB1562, G-12, H-8, C-20, FL-253, M-20, 8B4, 5B2, AH6, 6G3, and 6D11.

In other embodiments, a protein aggregate is an alpha-synuclein aggregate and epitope-binding agent binds to an epitope on the alpha-synuclein aggregate. Epitopes on alpha-synuclein aggregates and epitope-binding agents that bind thereto are known in the art. Non-limiting examples of epitope-binding agents that bind alpha-synuclein aggregate include 9B6, 14H2L1, 14HCLC, 4D6, 4B12, syn211, 24.8, 3H9, EP1646Y, 2A7, 5C2, 3B6, 2B2D1, AB138501, and 211.

In other embodiments, a protein aggregate is an htt aggregate and epitope-binding agent binds to an epitope on the htt aggregate. Epitopes on htt aggregates and epitope-binding agents that bind thereto are known in the art. Non-limiting examples of epitope-binding agents that bind htt aggregate include AB45169, AB9322, AB10514P, AB1594P, AB1772, EB06743, ABIN185394, ABIN374556, ABIN735983, ABIN250555, PA1706, PA1705, PA5-18374, sc-1458, sc-14514, sc-14516, sc-33724, or the epitopes recognized by the series of “MW” antibodies disclosed in Khoshnan et al. Methods Mol Biol. 2013; 1010:231-51 and Khoshnan et al. Methods Mol Biol. 2004; 277:87-102, each hereby incorporated by reference in its entirety. Non-limiting examples of suitable epitope-binding agents include the series of “MW” antibodies disclosed in Khoshnan et al. Methods Mol Biol. 2013; 1010:231-51 and Khoshnan et al. Methods Mol Biol. 2004; 277:87-102.

As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art (See, e.g. U.S. Pat. No. 7,939,313; herein incorporated by reference in its entirety).

An epitope-binding agent may be linked to a solid surface. Non-limiting examples of suitable surfaces include microwell plates, test tubes, beads, resins, microspheres, microparticles, nanoparticles, liposomes or and polymers. An epitope-binding agent may be attached to solid surface in a wide variety of ways, as will be appreciated by those in the art. An epitope-binding agent may either be synthesized first, with subsequent attachment to the substrate, or may be directly synthesized on the substrate. The substrate and the epitope-binding agent may be derivatized with chemical functional groups for subsequent attachment of the two. For example, the substrate may be derivatized with a chemical functional group including, but not limited to, amino groups, carboxyl groups, oxo groups, NHS-ester groups, malemide reactive groups, or thiol groups. Using these functional groups, the epitope-binding agent may be attached directly using the functional groups or indirectly using linkers. An epitope-binding agent may also be attached to a surface non-covalently. For example, a biotinylated epitope-binding agent may be prepared, which may bind to surfaces covalently coated with streptavidin, resulting in attachment. Alternatively, an epitope-binding agent may be synthesized on the surface using techniques such as photopolymerization and photolithography. Additional methods of attaching epitope-binding agents to solid surfaces and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, both of which are hereby incorporated by reference in their entirety).

An epitope-binding agent may be detectably labeled. As used herein, the term “label” refers to a protein, compound, chemical element or moiety that can be detected. One of the ways in which an epitope-binding agent of the present invention can be detectably labeled is by linking the same to an enzyme and use in an enzyme immunoassay (EIA) or enzyme-linked immunosorbent assay (ELISA). This enzyme, when subsequently exposed to its substrate, will react with the substrate generating a chemical moiety which can be detected, for example, by spectrophotometric, fluorometric or by visual means. Non-limiting examples of suitable enzymes include alkaline phosphatase or peroxidase. Another way in which an epitope-binding agent can be detectably labeled is by linking the same to a radioactive isotope and use of a radioimmunoassay (RIA). The radioactive isotope can be detected by such means as the use of a gamma counter or a scintillation counter or by autoradiography. Isotopes which are particularly useful for the purpose of the present invention are known in the art. It is also possible to label epitope-binding agents with a fluorescent compound. When the fluorescently labeled antibody is exposed to light of the proper wave length, its presence can then be detected due to fluorescence. Epitope-binding agents also can be detectably labeled by coupling to a chemiluminescent compound. The presence of the chemiluminescently labeled epitope-binding agent is then determined by detecting the presence of luminescence that arises during the course of a chemical reaction. A bioluminescent compound can also be used to label epitope-binding agents. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase (including split luciferase) and sequorin. An epitope-binding agent may also be labeled with biotin, avidin, stretpavidin, protein A, protein G, antibodies or fragments thereof, polyhistidine, Ni2+, Flag tags, or myc tags. Methods for detecting these labels are well known in the art.

Alternatively, an epitope-binding agent may intrinsically produce a detectable signal. A non-limiting examples includes an intrinsically fluorescent small molecule that selectively binds a protein aggregate.

Detection of a labeled epitope-binding agent (or an epitope-binding agent that intrinsically produces a detectable signal) can be accomplished by a scintillation counter, for example, if the detectable label is a radioactive gamma emitter, or by a fluorometer, for example, if the label is a fluorescent material. In the case of an enzyme label, the detection can be accomplished by colorimetric methods which employ a substrate for the enzyme. Detection can also be accomplished by visual comparison of the extent of enzymatic reaction of a substrate in comparison with similarly prepared standards.

### III. Method of Classifying a Protein Aggregate

The invention encompasses a method for classifying a protein aggregate. The protein aggregate may be completely or partially purified. Alternatively, the protein aggregate may be in a biological sample. The method may comprise (a) contacting a protein aggregate with a plurality of epitope-binding agents; (b) contacting the product of step (a) with a plurality of epitope-binding agents linked to a label (“labeled epitope-binding agents”) to form a labeled aggregate; (c) measuring the amount of detectable signal for each labeled aggregate; and (d) classifying the protein aggregate by assigning the protein aggregate to a discrete spatial location within a multivariate space. The terms “epitope-binding agent” and “protein aggregate” are described in detail above.

In some embodiments, a protein aggregate may be comprised of pathological protein associated with a neurodegenerative disease. In other embodiments, a protein aggregate may be comprised of pathological protein associated with a peripheral disease. In still other embodiments, a protein aggregate may be an amyloid and the amyloid may be associated with a central nervous system disease. In still different embodiments, a protein aggregate may be an amyloid and the amyloid may be associated with a peripheral disease. In yet other embodiments, a protein aggregate may be comprised of protein selected from the group consisting of prion protein, tau protein, alpha-synuclein protein, amyloid beta peptide, TDP-43, and htt. In different embodiments, a protein aggregate may be comprised a protein selected from the group consisting of amyloid beta peptide, PrP^{sc}, huntingtin, calcitonin, apolipoprotein A1, transthyretin, and cystatin. In still different embodiments, a protein aggregate may be comprised a protein selected from the group consisting of tau and prior protein.

### A. Contacting a Protein Aggregate with a Plurality of Epitope-Binding Agents

An aspect of the method comprises contacting a protein aggregate with x number of epitope-binding agents. The number of epitope-binding agents can and will vary depending, in part, upon the protein aggregate. Preferably, x≧3. For example, x may be an integer selected from the group consisting of 3, 4, 5, 6, 7, 8, 9, 10, 11, 12, 13, 14, 15, 16, 17, 18, 19, 20, 21, 22, 23, 24, 25, 26, 27, 28, 29, and 30. Alternatively, x may be a range selected from the group consisting of 3-5, 4-6, 5-7, 6-8, 7-9, 8-10, 3-7, 5-10, 3-20, 5-20, 10-20, 20-30, 30-40, 40-50, and 3-50. It is also preferable that the epitope-binding agents collectively bind at least three distinct epitopes. A “distinct epitope” may be an epitope that shares no amino acid identity with another epitope, or it may overlap with another epitope, provided that there is at least a single amino acid difference between the overlapping peptides. The use of two or more epitope-binding agents that bind to the same epitope is not detrimental to the method of the invention. Neither, however, does the use of two or more epitope-binding agents that bind to the same epitope necessarily improve the system. Accordingly, in certain embodiments, a method of the invention for classifying a protein aggregate comprises contacting a protein aggregate with x number of epitope-binding agents, wherein x≧3 and none of the epitope-binding agents bind the same epitope. In certain embodiments, each epitope-binding agent is linked to a label, a solid surface, or any combination thereof. Non-limiting examples of suitable labels include enzymes, radioactive isotopes, fluorescent compounds, chemical compounds, and bioluminescent proteins. Non-limiting examples of suitable solid surfaces include particles, beads, and resins. A “particle” refers to particles of varying size, typically nanoparticles and microparticles.

The choice of the epitope-binding agents will depend upon the protein aggregate. Any epitope-binding agent known to bind the protein aggregate may be used. Preferably, it is known that an epitope-binding agent binds at least 2, more preferably at least 3 or at least 4 or more conformers of the protein aggregate. The ideal binding agent will recognize a variety of conformers, but will exhibit different avidities for each. This enables a multiplex analysis to generate diversity and richness in terms of signal and facilitates parsing of different structures. In certain embodiments, it may be advantageous to select at least three epitope-binding agents whose binding would not be predicted to be sterically hindered by the others. For example, if available, selection of the three or more epitope-binding agents may be guided by models of the monomer and/or the protein aggregate in which the epitope(s) recognized by candidate epitope-binding agent(s) are mapped. Alternatively, choice of epitope-binding agents may be determined empirically. Epitopes may or may not be distributed throughout the linear polypeptide sequence, based upon the proteins tertiary structure. In the examples described herein, epitopes were distributed throughout the tau molecule.

In some embodiments, each of the three or more epitope-binding agents is independently selected from the group consisting of an antibody and an aptamer. For example, if x is 3, then all three epitope-binding agents can be an antibody, all three epitope-binding agents can be an aptamer, 2 epitope-binding agents can be an antibody and 1 epitope-binding agent can be an aptamer, or 2 epitope-binding agents can be an aptamer and 1 epitope-binding agent can be an antibody. In preferred embodiments, each of the epitope-binding agents is linked to a solid surface. In exemplary embodiments, the solid surface is a particle or a resin.

Contacting a protein aggregate with a plurality of epitope-binding agents generally involves providing a mixture comprising a buffer, the protein aggregate and one or more of the epitope-binding agents and incubating the mixture, optionally with mixing, for a period of time long enough for the epitope-binding agent to bind to its cognate epitope on the protein aggregate if present. The amount of the epitope-binding agent and protein aggregate can and will vary, provided that the amount of the epitope-binding agent is non-saturating. Stated another way, the epitope-binding agent is exposed to an excess of protein aggregate. The molar ratio of aggregate to epitope-binding agent may be about 2:1 to about 10⁶:1 or more, about 5:1 to about 10⁶:1 or more, about 10:1 to about 10⁶:1 or more, about 50:1 to about 10⁶:1 or more, or about 10²:1 to about 10⁶:1 or more. Use of a non-saturating amount of an epitope-binding agent in this step is necessary to ensure that there are epitopes available on the surface of the protein aggregate for the epitope-binding agents in step (b) to bind. This step may comprise one or more reactions, depending upon whether there are separate mixtures for each epitope-binding agent or the epitope-binding agents are provided in combination. The length of time will vary, in part, depending upon the incubation temperature. Suitable reaction temperatures, durations of incubation and buffers are well-known in the art, and may be optimized by routine experimentation. Following incubation, the mixture may optionally be further processed. For example, excess epitope-binding agent may be removed, protein aggregate bound to epitope-binding may be washed and/or resuspended in a new buffer, or combinations thereof. Preferably, labeled aggregate is washed to remove to excess epitope-binding agent. These steps may be facilitated by using an epitope-binding agent linked to a solid surface.

### B. Contacting the Product of Step (a) with a Plurality of Labeled Epitope-Binding

Another aspect of the method comprises contacting the product of step (a) with y number of a epitope-binding agents linked to a label (“labeled epitope-binding agents”) to form y types of labeled aggregate, wherein y=x (as defined in Section III(A)) and the labeled epitope-binding agents of step (b) and the epitope-binding agents from step (a) collectively recognize the same epitopes. As illustrated in FIG. 12A, in which the method is exemplified with a single antibody, if a monomer is bound by step (a), its epitope is occupied and the labeled epitope-binding agent doesn't bind. However, if a multimer is bound in step (a), multiple epitopes remain available and the labeled epitope-binding agent binds.

In some embodiments, the epitope-binding agents of step (a) and epitope-binding agents of step (b) are the same, with the provisio that the epitope-binding agents from step (b) are linked to a label. As a non-limiting example, if the epitope-binding agents from step (a) are antibody 1, antibody 2 and antibody 3, then the labeled epitope-binding agents of step (b) may be antibody 1 linked to a label, antibody 2 linked to a label, and antibody 3 linked to a label. In other embodiments, despite collectively recognizing the same epitopes, the epitope-binding agents of step (a) and epitope-binding agents of step (b) are the different. As a non-limiting example, if the epitope-binding agents from step (a) are antibody 1, antibody 2 and antibody 3, then the labeled epitope-binding agents of step (b) may be antibody 4 linked to a label, antibody 5 linked to a label, and antibody 6 linked to a label, provided the antibodies of step (b) collectively recognize the same epitopes as the antibodies of step (a). Alternatively, if the epitope-binding agents from step (a) are antibody 1, antibody 2 and antibody 3, then the labeled epitope-binding agents of step (b) may be aptamer 1 linked to a label, aptamer 2 linked to a label, and aptamer 3 linked to a label, provided the aptamers of step (b) collectively recognize the same epitopes as the antibodies of step (a). One skilled in the art can readily envision other embodiments, given the disclosures of Section II and III.

Suitable labels are described in detail in Section II above. Non-limiting examples of suitable labels include enzymes, radioactive isotopes, fluorescent compounds, chemical compounds, and bioluminescent proteins. Choice of label can and will vary depending upon detection method and method to measure the detectable signal. If the labeled epitope-binding agents are provided in combination, then none of the labels of the epitope-binding agents can be the same. Rather, the labels must be able to be distinguishable by the detection method. For example, fluorescent compounds may be used, provided compounds' emission spectra can be resolved. Typically, a combination of fluorescent compounds may be selected where none of the compounds' emission spectra significantly overlap. In addition, when the association of different epitope-binding agents with an aggregate is measured via FRET between labels on the epitope-binding agents, suitable FRET pairs should be selected. If the labeled epitope-binding agents are not provided in combination, same or different labels may be used. In preferred embodiments, the label is a fluorescent compound. Suitable fluorescent compounds are well known in the art, as are methods for choosing fluorescent compounds to be used in combination and/or fluorescent compounds that are suitable for FRET.

Contacting the product of step (a) with y number of labeled epitope-binding agents to form y types of labeled aggregate generally involves providing a mixture comprising a buffer, the product of step (a) and one or more of the y number of epitope-binding agents and incubating the mixture, optionally with mixing, for a period of time long enough for the epitope-binding agent(s) to bind to its cognate epitope on the protein aggregate if present. The amount of the epitope-binding agent and protein aggregate can and will vary, but it is generally desirable to have an excess of protein aggregate to epitope-binding agent. This step may comprise one or more reactions, depending upon whether there are separate mixtures for each labeled epitope-binding agent or the labeled epitope-binding agents are provided in combination. The length of time will vary, in part, depending upon the incubation temperature. Suitable reaction temperatures, durations of incubation and buffers are well-known in the art, and may be optimized by routine experimentation. Following incubation, the mixture may optionally be further processed. For example, excess labeled epitope-binding agent may be removed, labeled aggregate may be washed and/or resuspended in a new buffer, or combinations thereof. Preferably, labeled aggregate is washed to remove to excess labeled epitope-binding agent.

### C. Measuring the Amount of Label for Each Type of Labeled Aggregate

Another aspect of the invention comprises measuring the amount of label for each type of labeled aggregate, wherein the amount of label is directly proportional to binding avidity of the labeled epitope-binding agent for the protein aggregate. Briefly, each labeled aggregate in the product of step (b) provides a measure of label intensity reflecting the number of labeled epitope-binding agents that are bound to the protein aggregate. In some embodiments, a measurement may be taken at a population level for each type of labeled aggregate. In other embodiments, a measurement may be taken at the level of an individual aggregate. Suitable detection methods based on the type of label are described above in Section II. The amount of label may be measured as the amount of energy transferred between two labels (i.e. FRET measurement) or amount of energy emitted by the label.

When measurements are taken at a population level, two parameters are derived from the measurement of label intensity: 1) Percent positivity, i.e. the percentage of labeled protein aggregates in the product from step (b) with fluorescence above background, and 2) median fluorescence intensity, i.e. the median fluorescence of the population of positive labeled protein aggregates from step (b). The natural log of the product of these two parameters may be calculated for each labeled epitope-binding agent. This calculation is directly proportional to binding avidity of the labeled epitope for the protein aggregate present in the product of step (b).

In a specific embodiment, the label is a fluorescent compound and the amount of label is detected by flow cytometry. Briefly, the product of step (b) may be used to perform flow cytometry. Forward scatter area, forward scatter height, side scatter area, side scatter height, fluorescence, and combinations thereof may be measured. If the product from step (b) contains a combination of labels in a single sample, the combination(s) which can be used may depend, in part, on the wavelength of the lamp(s) or laser(s) used to excite the fluorochromes and on the detectors available. Events may be gated for size as appropriate. For example, gating on size may be used to exclude sub- or multi-particles/resins when an epitope-binding agent is linked to a solid support. Samples containing no antigen may be used to create an arbitrary threshold of fluorescence positivity. Each labeled aggregate in the product of step (b) provides a measure of fluorescence intensity reflecting the number of labeled epitope-binding agents that are bound to the protein aggregate. Two parameters are derived from measurement of fluorescence intensity: 1) Percent positivity, i.e. the percentage of labeled protein aggregates in the product from step (b) with fluorescence above background, and 2) median fluorescence intensity, the median fluorescence of the population of positive labeled protein aggregates from step (b). The natural log of the product of these two parameters may be calculated for each labeled epitope-binding agent.

In another specific embodiment, fluorescence correlation spectroscopy, or other microfluidic approaches that enable observations of individual particles and their fluorescence levels can be used.

When a measurement is taken at the level of an individual aggregate, a microfluidic approach would be employed to define the size of each individual labeled aggregate in addition to a measurement of fluorescent intensity. The relative binding of a given epitope-binding agent to an aggregate would be determined (i.e. its avidity) based on the signal intensity recorded for that particular labeled aggregate. Subsequently, after measuring hundreds or thousands of individual events, a multivariate profile of the sample that incorporated distribution of the labeled aggregate size and the relative labeling of each would be used to characterize aggregate structures.

### D. Classifying the Protein Aggregate by Assigning the Protein Aggregate to a Discrete Spatial Location within a Multivariate Space

Another aspect of the invention comprises classifying a protein aggregate by assigning the protein aggregate to a discrete spatial location within a multivariate space. The multivariate space has n number of axes, wherein n=y (as defined in Section III(B)) and each axis corresponds to a labeled epitope-binding agent, or, in the case of a size analysis of an aggregate, the distribution of aggregates of particular sizes, and wherein a coordinate along the axis is the binding avidity (as determined in Section III(C)). For any two aggregates that significantly differ in their binding profile to the labeled epitope-binding agents, the aggregates will occupy discrete locations within the multivariate space. Conversely, for any two aggregates that have significantly similar binding profiles to the labeled epitope-binding agents, the aggregates will co-localized within the multivariate space.

### E. Preferred Embodiments

In a specific embodiment, the present invention provides a method for classifying an amyloid, the method comprising (a) contacting the amyloid with x number of anti-amyloid epitope-binding agents linked to a solid support, wherein x≧3 and none of the epitope-binding agents bind the same epitope; (b) contacting the product of step (a) with y number of anti-amyloid epitope-binding agents linked to a label (“labeled epitope-binding agents”) to form y types of labeled amyloids, wherein y=x and the labeled anti-amyloid epitope-binding agents of step (b) and the anti-amyloid epitope-binding agents from step (a) collectively recognize the same epitopes; (c) measuring the amount of label for each type of labeled amyloid, wherein the amount of the label is directly proportional to binding avidity of the labeled anti-amyloid epitope-binding agent for the amyloid; and (d) classifying the amyloid by assigning the amyloid to a discrete spatial location within a multivariate space having n number of axes, wherein n=y and each axis corresponds to a labeled anti-amyloid epitope-binding agent, and wherein a coordinate along the axis is the binding avidity calculated in step (c). The amyloid may be comprised of a protein selected from the group consisting of amyloid beta peptide, prion protein, huntingtin, calcitonin, apolipoprotein A1, IAPP, AANF, transthyretin, tau, cystatin, serum amyloid, medin, prolactin, lysozyme, Beta 2 microglobulin, gelsolin, keratoepithelin, immunoglobulin light chain AL, and S-IBM, and the epitope binding agent may be selected from the group consisting of an antibody, an aptamer, a protein, a lipid, and a small molecule. Step (a) may further comprise contacting a biological sample, or fraction thereof, comprising an amyloid with x number of epitope-binding agents linked to a solid support, wherein x≧3 and none of the epitope-binding agents bind the same epitope.

In another specific embodiment, the present invention provides a method for classifying a tau aggregate, the method comprising (a) contacting the tau aggregate with x number of anti-tau antibodies linked to a solid support, wherein x≧3 and none of the antibodies bind the same epitope; (b) contacting the product of step (a) with y number of anti-tau antibodies linked to a label (“labeled antibodies”) to form y types of labeled tau aggregates, wherein y=x and the labeled anti-tau antibodies of step (b) and the anti-tau antibodies from step (a) collectively recognize the same epitopes; (c) measuring the amount of label for each type of labeled tau aggregate, wherein the amount of the label is directly proportional to binding avidity of the labeled anti-tau antibody for the tau aggregate; and (d) classifying the tau aggregate by assigning the tau aggregate to a discrete spatial location within a multivariate space having n number of axes, wherein n=y and each axis corresponds to a labeled anti-tau antibody, and wherein a coordinate along the axis is the binding avidity calculated in step (c). Step (a) may further comprise contacting a biological sample, or fraction thereof, comprising a tau aggregate with x number of anti-tau antibodies linked to a solid support, wherein x≧3 and none of the antibodies bind the same epitope.

In a specific embodiment, the present invention provides a method for classifying a tau aggregate, the method comprising (a) contacting the tau aggregate with x number of anti-tau aptamers linked to a solid support, wherein x≧3 and none of the aptamers bind the same epitope; (b) contacting the product of step (a) with y number of anti-tau aptamers linked to a label (“labeled aptamers”) to form y types of labeled tau aggregates, wherein y=x and the labeled anti-tau aptamers of step (b) and the anti-tau aptamers from step (a) collectively recognize the same epitopes; (c) measuring the amount of label for each type of labeled tau aggregate, wherein the amount of the label is directly proportional to binding avidity of the labeled anti-tau aptamer for the tau aggregate; and (d) classifying the tau aggregate by assigning the tau aggregate to a discrete spatial location within a multivariate space having n number of axes, wherein n=y and each axis corresponds to a labeled anti-tau aptamer, and wherein a coordinate along the axis is the binding avidity calculated in step (c). Step (a) may further comprise contacting a biological sample, or fraction thereof, comprising a tau aggregate with x number of anti-tau aptamers linked to a solid support, wherein x≧3 and none of the aptiamers bind the same epitope.

In a specific embodiment, the present invention provides a method for classifying a prion aggregate, the method comprising (a) contacting the prion aggregate with x number of anti-prion antibodies linked to a solid support, wherein x≧3 and none of the antibodies bind the same epitope; (b) contacting the product of step (a) with y number of anti-prion antibodies linked to a label (“labeled antibodies”) to form y types of labeled prion aggregates, wherein y=x and the labeled anti-prion antibodies of step (b) and the anti-prion antibodies from step (a) collectively recognize the same epitopes; (c) measuring the amount of label for each type of labeled prion aggregate, wherein the amount of the label is directly proportional to binding avidity of the labeled anti-prion antibody for the prion aggregate; and (d) classifying the prion aggregate by assigning the prion aggregate to a discrete spatial location within a multivariate space having n number of axes, wherein n=y and each axis corresponds to a labeled anti-prion antibody, and wherein a coordinate along the axis is the binding avidity calculated in step (c). Step (a) may further comprise contacting a biological sample, or fraction thereof, comprising a prion aggregate with x number of anti-prion protein antibodies linked to a solid support, wherein x≧3 and none of the antibodies bind the same epitope.

In a specific embodiment, the present invention provides a method for classifying a prion aggregate, the method comprising (a) contacting the prion aggregate with x number of anti-prion aptamers linked to a solid support, wherein x≧3 and none of the aptamers bind the same epitope; (b) contacting the product of step (a) with y number of anti-prion aptamers linked to a label (“labeled aptamers”) to form y types of labeled prion aggregates, wherein y=x and the labeled anti-prion aptamers of step (b) and the anti-prion aptamers from step (a) collectively recognize the same epitopes; (c) measuring the amount of label for each type of labeled prion aggregate, wherein the amount of the label is directly proportional to binding avidity of the labeled anti-prion aptamer for the prion aggregate; and (d) classifying the prion aggregate by assigning the prion aggregate to a discrete spatial location within a multivariate space having n number of axes, wherein n=y and each axis corresponds to a labeled anti-prion aptamer, and wherein a coordinate along the axis is the binding avidity calculated in step (c). Step (a) may further comprise contacting a biological sample, or fraction thereof, comprising a prion aggregate with x number of anti-prion protein aptamers linked to a solid support, wherein x≧3 and none of the aptamers bind the same epitope.

In a specific embodiment, the present invention provides a method for classifying an amyloid beta peptide aggregate, the method comprising (a) contacting the amyloid beta peptide aggregate with x number of anti-amyloid beta peptide antibodies linked to a solid support, wherein x≧3 and none of the antibodies bind the same epitope; (b) contacting the product of step (a) with y number of anti-amyloid beta peptide antibodies linked to a label (“labeled antibodies”) to form y types of labeled amyloid beta peptide aggregates, wherein y=x and the labeled anti-amyloid beta peptide antibodies of step (b) and the anti-amyloid beta peptide antibodies from step (a) collectively recognize the same epitopes; (c) measuring the amount of label for each type of labeled amyloid beta peptide aggregate, wherein the amount of the label is directly proportional to binding avidity of the labeled anti-amyloid beta peptide antibody for the amyloid beta peptide aggregate; and (d) classifying the amyloid beta peptide aggregate by assigning the amyloid beta peptide aggregate to a discrete spatial location within a multivariate space having n number of axes, wherein n=y and each axis corresponds to a labeled anti-amyloid beta peptide antibody, and wherein a coordinate along the axis is the binding avidity calculated in step (c). Step (a) may further comprise contacting a biological sample, or fraction thereof, comprising an amyloid beta peptide aggregate with x number of anti-amyloid beta peptide antibodies linked to a solid support, wherein x≧3 and none of the antibodies bind the same epitope.

In a specific embodiment, the present invention provides a method for classifying an amyloid beta peptide aggregate, the method comprising (a) contacting the amyloid beta peptide aggregate with x number of anti-amyloid beta peptide aptamers linked to a solid support, wherein x≧3 and none of the aptamers bind the same epitope; (b) contacting the product of step (a) with y number of anti-amyloid beta peptide aptamers linked to a label (“labeled aptamers”) to form y types of labeled amyloid beta peptide aggregates, wherein y=x and the labeled anti-amyloid beta peptide aptamers of step (b) and the anti-amyloid beta peptide aptamers from step (a) collectively recognize the same epitopes; (c) measuring the amount of label for each type of labeled amyloid beta peptide aggregate, wherein the amount of the label is directly proportional to binding avidity of the labeled anti-amyloid beta peptide aptamer for the amyloid beta peptide aggregate; and (d) classifying the amyloid beta peptide aggregate by assigning the amyloid beta peptide aggregate to a discrete spatial location within a multivariate space having n number of axes, wherein n=y and each axis corresponds to a labeled anti-amyloid beta peptide aptamer, and wherein a coordinate along the axis is the binding avidity calculated in step (c). Step (a) may further comprise contacting a biological sample, or fraction thereof, comprising an amyloid beta peptide aggregate with x number of anti-amyloid beta peptide aptamers linked to a solid support, wherein x≧3 and none of the aptamers bind the same epitope.

### IV. Method for Comparing the Similarity of Two or More Protein Aggregates

The invention encompasses a method for comparing the similarity of two or more protein aggregates. Each protein aggregate may be completely or partially purified. Alternatively, each protein aggregate may be in the same or different biological sample. The method may comprise (a) classifying each protein aggregate; and (b) calculating a degree of similarity between the protein aggregates. Methods for classifying a protein aggregate are described above in Section III.

Any suitable statistical method known in the art may be used to calculate a degree of similarity between the protein aggregates. For example, similarity may be calculated by determining a Euclidean distance between all pairwise combinations of samples and using a suitable statistical algorithm to cluster the results and bind the samples into discrete groups. Such algorithms are well known in the art and include, but is not limited to, K-means clustering. K-means clustering uses an algorithm to determine in an iterative series of analyses the most optimal way to partition a number of observations into K clusters, in which each observation belongs to the cluster with the nearest mean. Determining similarity by calculating a Euclidean distance between all pairwise combinations of samples, or by using similar methods, is a suitable approach when the total amount of protein aggregate between samples does not significantly vary. Similarity may also be calculated by determining correlation coefficients between the binding signals across all protein aggregates and epitope-binding agents to determine which samples are most similar to one another. Determining similarity by calculating correlation coefficients, or by using similar methods, is a suitable approach when the total amount of protein aggregate may vary between samples.

### V. Other Aspects

In another aspect, the present invention encompasses a method for assigning a location in a multivariate space to a disease associated with a protein aggregate, preferably an amyloid. The method may comprise (a) obtaining a sample from a subject diagnosed with a disease associated with a protein aggregate, (b) classifying the protein aggregate as described in Section III, and (c) assigning the spatial location of the protein aggregate within the multivariate space to the disease of the subject. Diseases associated with protein aggregates are described above in Section I. Accordingly, once a location in the multivariate space is assigned to a disease (or more than one disease), any sample comprising a protein aggregate classified to a location that is similar may be correlated with the disease. Similarity may be determined as described above in Section IV. Without wishing to be bound by theory, a spatial location may be assigned to more than one disease. As a non-limiting example, one conformer of a tau aggregate may be associated with two different forms a tauopathy but not associated with a third.

In some embodiments, the protein is tau and the disease associate with tau is a tauopathy. Non-limiting examples of tauopathies include progressive supranuclear palsy, dementia pugilistica, frontotemporal dementia and parkinsonism linked to chromosome 17, Lytico-Bodig disease, tangle-predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, argyrophilic grain disease (AGD), Frontotemporal lobar degeneration, Alzheimer's Disease, and frontotemporal dementia.

In other embodiments, the protein is prion protein and the disease associated with prion protein is selected from the group consisting of scrapie, bovine spongiform encephalopathy, transmissible mink encephalopathy, chronic wasting disease, feline spongiform encephalopathy, exotic ungulate encephalopathy, Creutzfeldt-Jakob diseases, Gerstmann-Straussler-Scheinker syndrome, fatal familial insomnia, and Kuru.

In other embodiments, the protein is amyloid beta protein and the disease associated with prion protein is selected from the group consisting of Alzheimer's disease, Lewy body disease, cerebral amyloid angiopathy, inclusion body myositis and traumatic brain injury.

This invention could be used to diagnose subjects with a neurological disease characterized by pathological protein aggregation.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and the scope of the invention. Therefore, all matter set forth or shown in the accompanying drawings is to be interpreted as illustrative and not in a limiting sense.

### EXAMPLES

The following examples illustrate various iterations of the invention.

### Example 1

### Microsphere-Based Antibody Sandwich

Method:

StrepAvidin microspheres are coated with a biotinylated monoclonal anti-tau antibody and incubated with a saturating amount of brain homogenate from a tauopathy patient. The microspheres are then washed and incubated with the same monoclonal antibody, labeled with a fluorescent dye for detection. This antibody sandwich excludes monomeric tau from detection as monomeric tau contains only one epitope for any particular monoclonal antibody. After incubation, the microspheres are washed and passed through a flow cytometer.

Analysis:

Each microsphere provides a measure of fluorescence intensity reflecting the number of antibodies that are bound to the tau on the microsphere. Two parameters are derived from the fluorescence intensity of a population: 1) Percent positivity, the percentage of microspheres with fluorescence above background, and 2) Median fluorescence intensity, the median fluorescence of the population of positive microspheres. The product of these two parameters is calculated for each antibody with respect to each brain. K-means analyses are used to cluster brains based on similarity of binding to the entire panel of antibodies.

High Throughput Capability:

Rather than incubate each antibody-antigen combination separately, we can simplify the process by including four detection antibodies (tagged to four distinct fluorescent dyes) and four microsphere sizes, each corresponding to one of the four antibodies, per reaction.

### Example 2

### Fingerprinting Free Flowing Conformers by Flow Cytometry

Method:

Tau is immunoprecipitated from the brain homogenate of a tauopathy patient using a polyclonal mixture of antibodies to ensure complete depletion. The (nearly) pure tau is incubated with a polyclonal anti-tau antibody and a monoclonal anti-tau antibody at a very dilute concentration (10 ug total protein per mL). The polyclonal and monoclonal antibodies are tagged with distinct fluorescent dyes for discrimination. After incubation, the tau+antibody solution is passed through the flow cytometer. Gain settings are critical, as they must be sensitive enough to detect large protein complexes while gating out monomeric protein.

Analysis:

Two measures of fluorescence intensity are provided for every event, one corresponding to the polyclonal antibody and one corresponding to the monoclonal antibody. The polyclonal antibody serves as a proxy for size, as it should bind to multiple epitopes throughout the protein. Plotting the monoclonal fluorescence on one axis and the polyclonal fluorescence on the other axis of a two-dimensional plot thus allows us to visualize antibody binding per unit of tau. This enables the detection of multiple strains within a sample, as distinct protein conformations are likely to have distinct binding patterns for one or more antibodies. Matlab is used to compare each voxel on one plot to the corresponding voxel on another plot in order to cluster samples based on similarity of antibody binding. Once again, several monoclonal antibodies can be simultaneously incubated with one sample as long as they are tagged with distinct fluorescent dyes.

### Example 3

### Discrimination of Strains of Tau Aggregates in Monoclonal Cell Lines

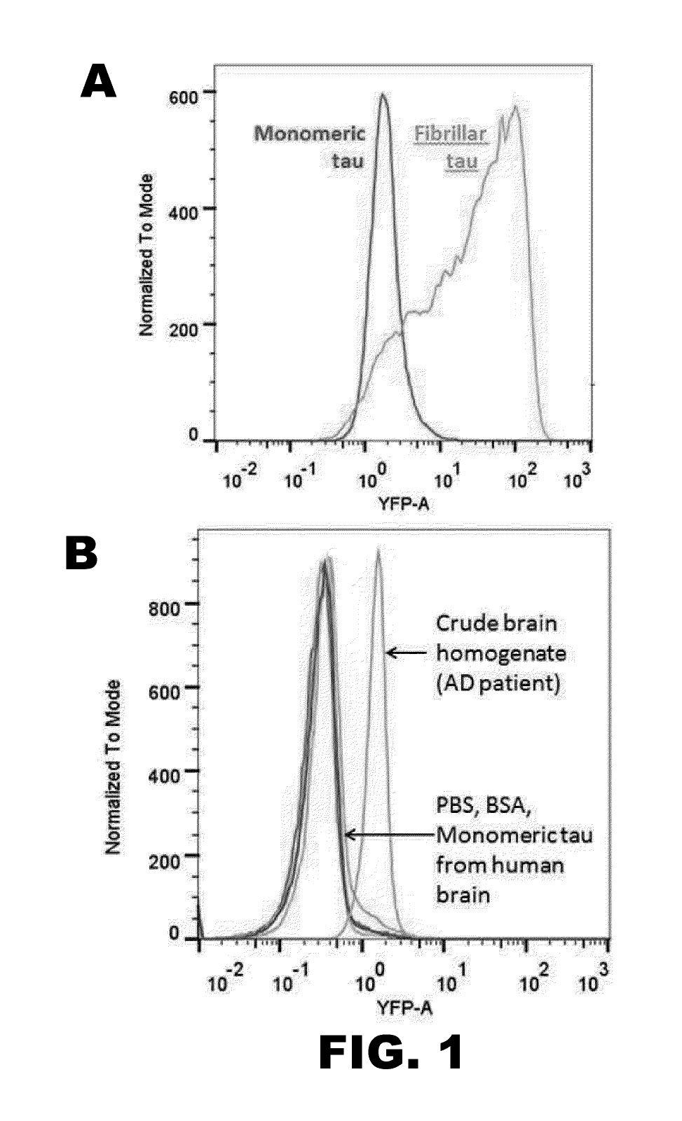

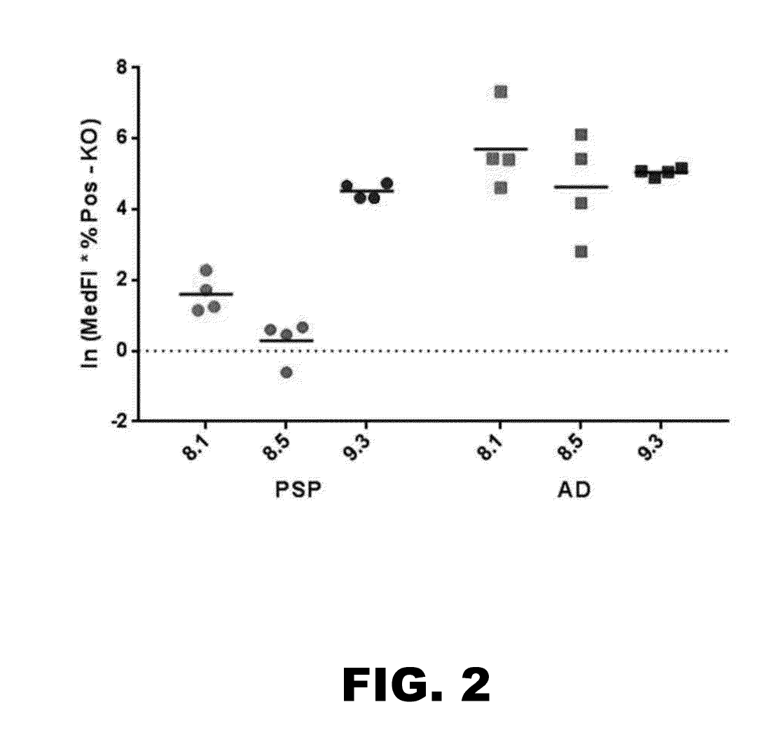

Various monoclonal cell lines have been shown to stably propagate distinct strains of tau inclusions via a panel of biochemical assays. The fingerprinting method described above is able to replicate the observed differences and predict the presence of multiple strains within a cell line, thus enhancing the original work. See FIG. 1 and FIG. 2.

### Example 4

### Classifying a Subject Using the Sandwich System

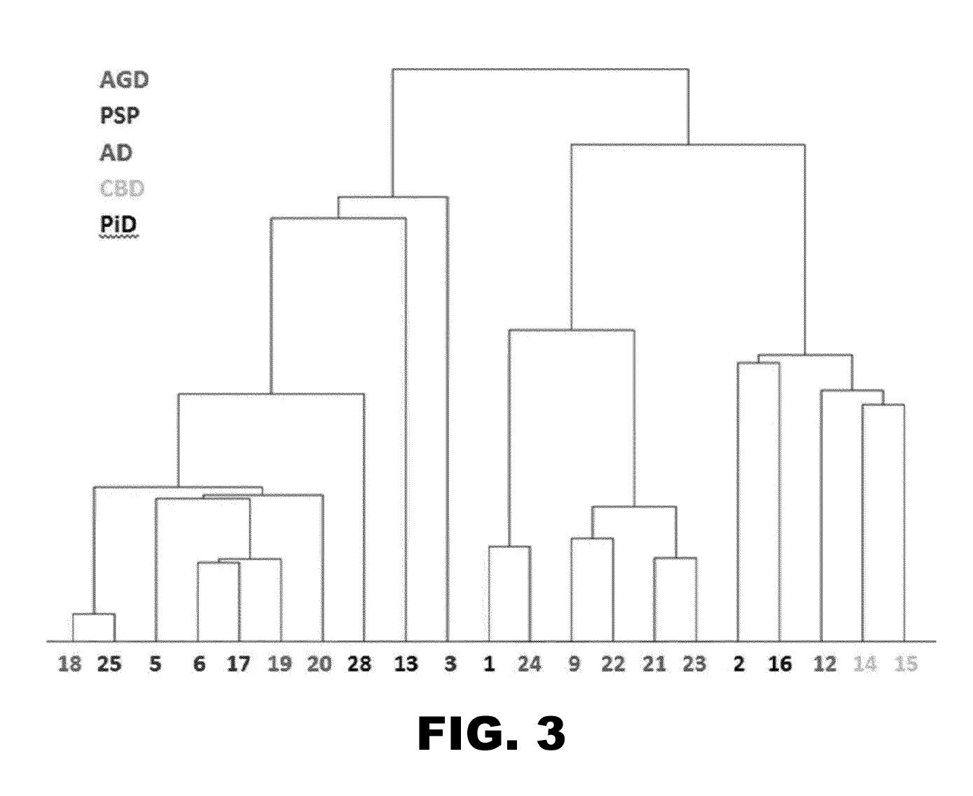

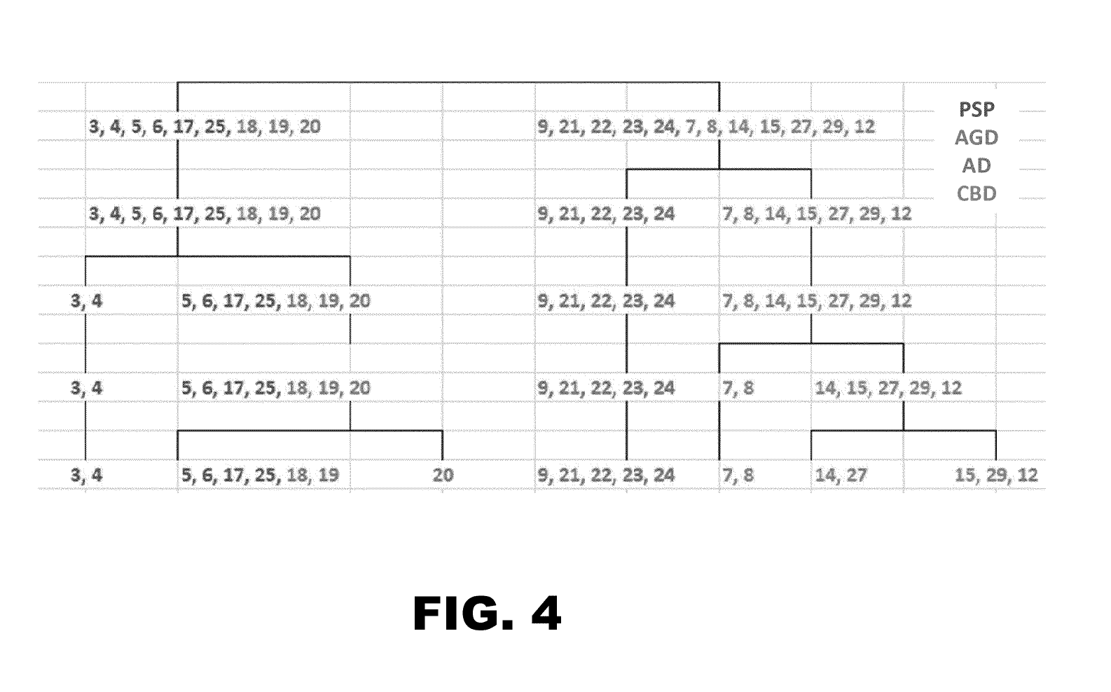

Brain homogenates from twenty one patients diagnosed with four distinct tauopathies (AD, AGD, CBD, and PSP) were characterized using the microsphere-based invention. The patients were grouped mostly by their neuropathological diagnoses. Interestingly, the clustering was consistent between two separate analyses using two mutually exclusive sets of antibodies. This demonstrates the power of multidimensional structure mapping and suggests that patient/disease discrimination does not rely on any particular antibody. Finally, this method has the potential to predict clinical outliers, which would provide insight into the differences that exist within a disease (e.g. differential rates of progression). See FIG. 3 and FIG. 4.

### Example 5

### Classifying a Subject Using the Fingerprint System

Preliminary work has shown that tau aggregates immunoprecipitated from two tauopathy patients can be visualized and discriminated from each other and from Htt aggregates (used as a negative control). This work is being continued in order to group patients by aggregate structure, which is reflected in antibody-binding patterns. Importantly, the fingerprinting invention allows for the detection of multiple strains within a patient sample and in theory can be applied to tau aggregates in the periphery. Similar methods may be used to detect aggregates from peripheral tissues (e.g. ISF, CSF, blood, serum, plasma), as well as other types of protein aggregates in addition to tau aggregates.

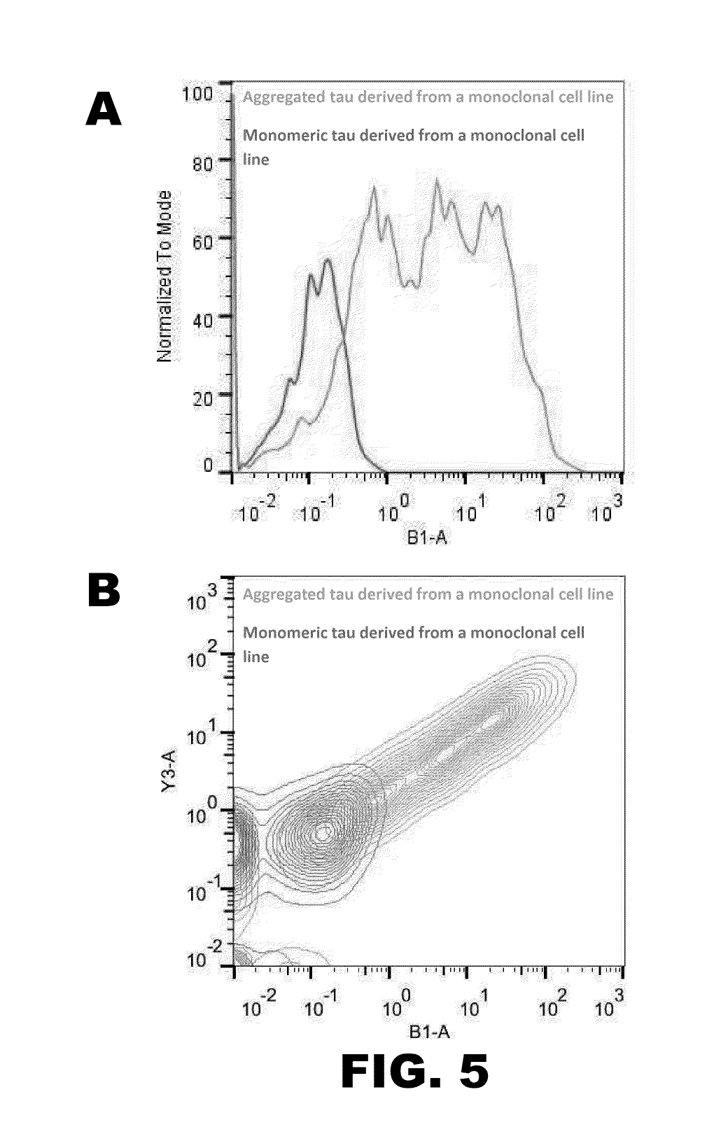

Aggregated tau may be discriminated from monomeric tau using the fingerprint system (FIG. 5). Two monoclonal cell lines containing equivalent amounts of tau RD fused to YFP were lysed and incubated with a monoclonal anti-tau antibody conjugated to a fluorescent dye. One cell line contained only diffuse tau RD-YFP (red) while the other stably propagated tau aggregates (blue). The solution was diluted and passed through a flow cytometer such that each event detected would provide a measure of both YFP and antibody fluorescence. The monomeric cell line displayed one YFP peak on the lower end of the fluorescence spectrum, indicating homogeneity in size (left). The cell line containing aggregated tau, however, displayed a range of YFP fluorescence, indicating a larger range of sizes. Plotting YFP fluorescence against antibody fluorescence shows one discrete level of antibody binding in the monomeric cell line, as expected (right). The cell line containing aggregated tau shows a logarithmic relationship between YFP fluorescence and antibody fluorescence, indicating that more antibodies bind to larger tau aggregates. Importantly, the relationship between antibody fluorescence and YFP fluorescence is consistent throughout the population, suggesting the presence of a single conformation of tau. This has been confirmed both biochemically and morphologically.

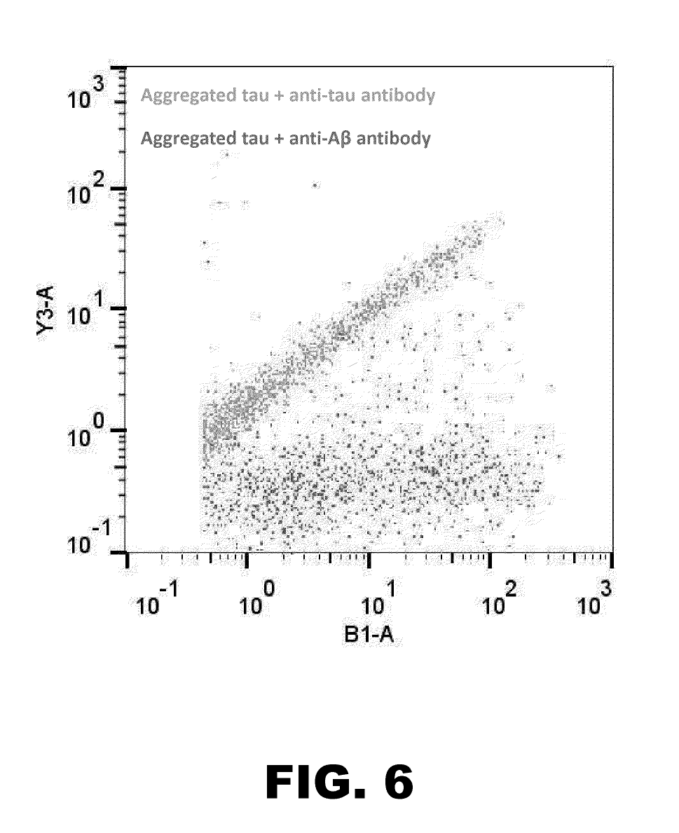

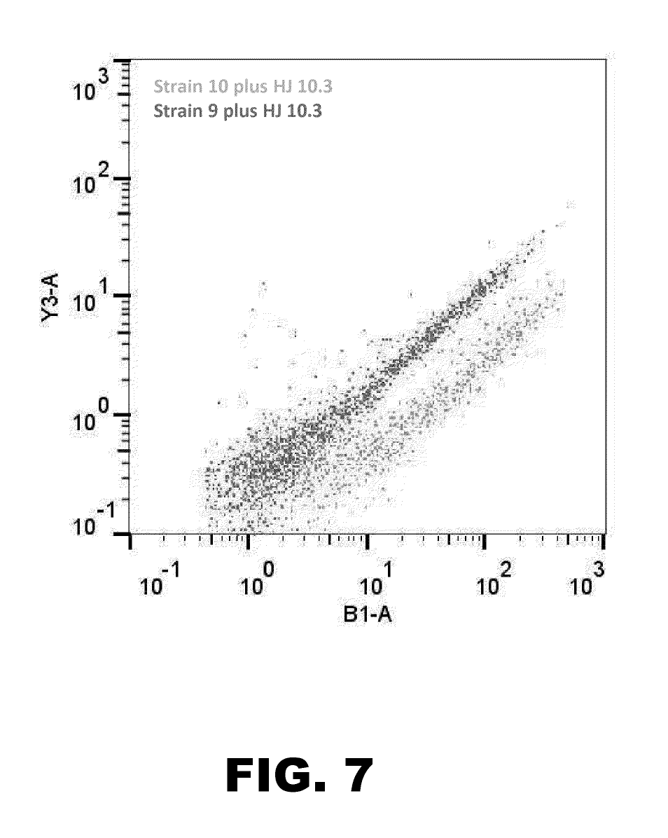

Anti-tau antibody binding is protein specific (FIG. 6). To test for the possibility of system artifact, aggregated tau derived from the monoclonal cell line described previously was incubated with either an anti-tau antibody or an anti-Aβ antibody, both conjugated to a fluorescent dye. As expected, the anti-tau antibody displayed greater binding to increasing aggregate sizes, while the anti-Aβ antibody did not. This data suggests that dual fluorescence positivity is not a result of coincidence, but of true antibody-antigen binding. Background positivity can be reduced approximately threefold by conjugating the same antibody to two distinct fluorescent dyes and gating for dual positivity among antibody fluorescence.

Two cell lines produce distinct conformation of tau (FIG. 7). Two monoclonal cell lines propagating distinct conformations of tau (as determined by morphological and biochemical assays) were lysed and incubated with a monoclonal antibody conjugated to a fluorescent tag. The strains show differential antibody binding per unit of tau, suggesting that there are fewer epitopes spatially available in one conformation. By itself, this data can conclude that the conformers in each cell line are structurally different.

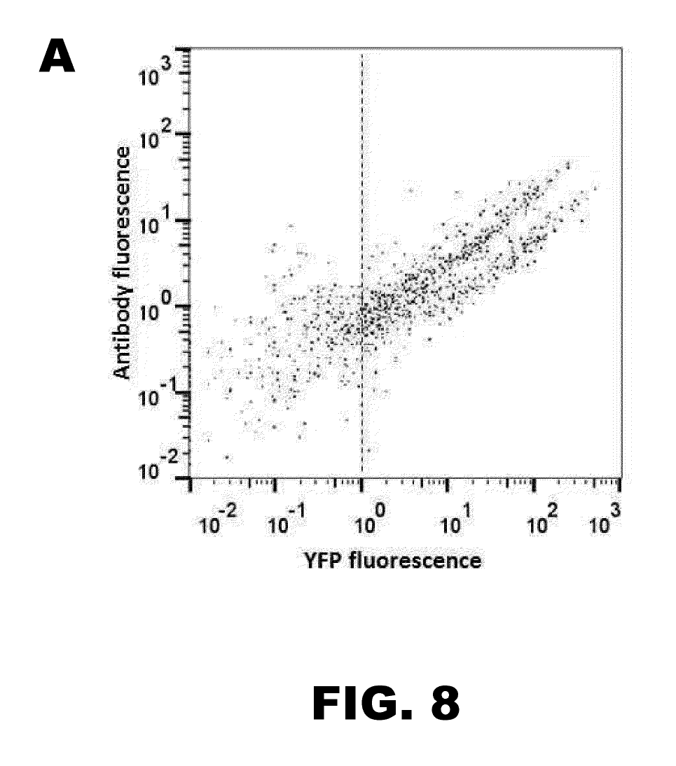

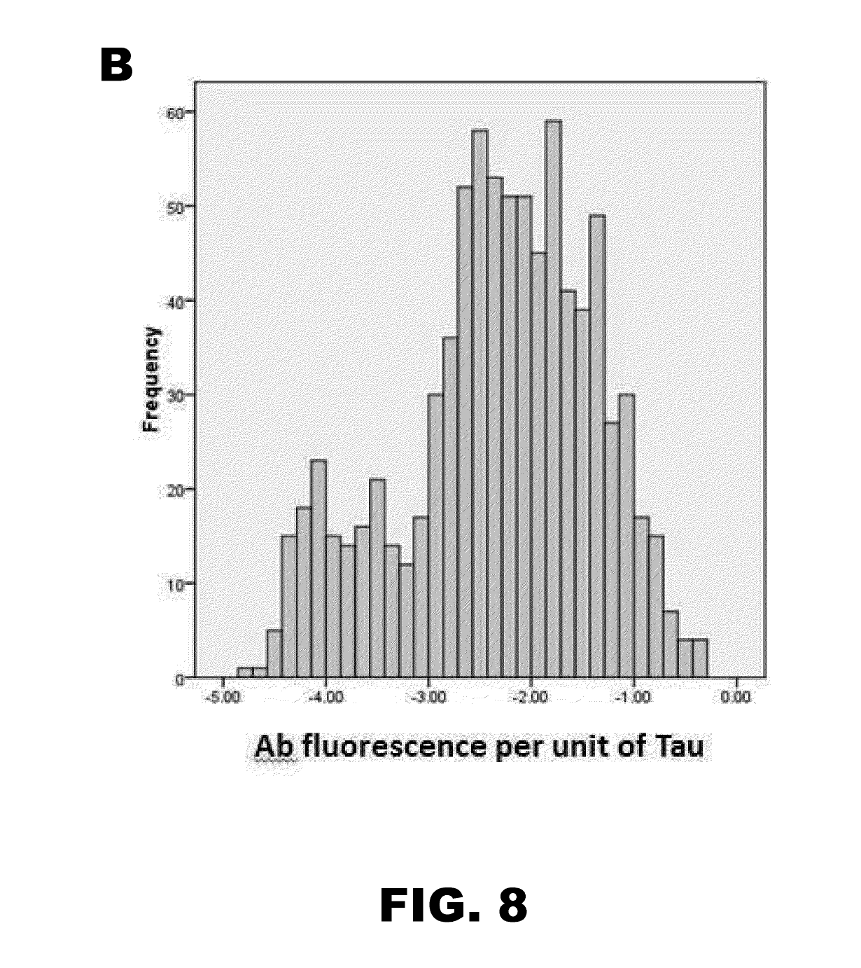

Multiple strains within the same sample may be discriminated (FIG. 8). The two strains described previously (9 and 10) were incubated in the same sample along with a monoclonal antibody that shows differential binding patterns to each strain. The ratio of antibody fluorescence per unit of tau was calculated for every event and plotted in a frequency histogram (right). Each of the two peaks in the histogram corresponds to an individual strain.

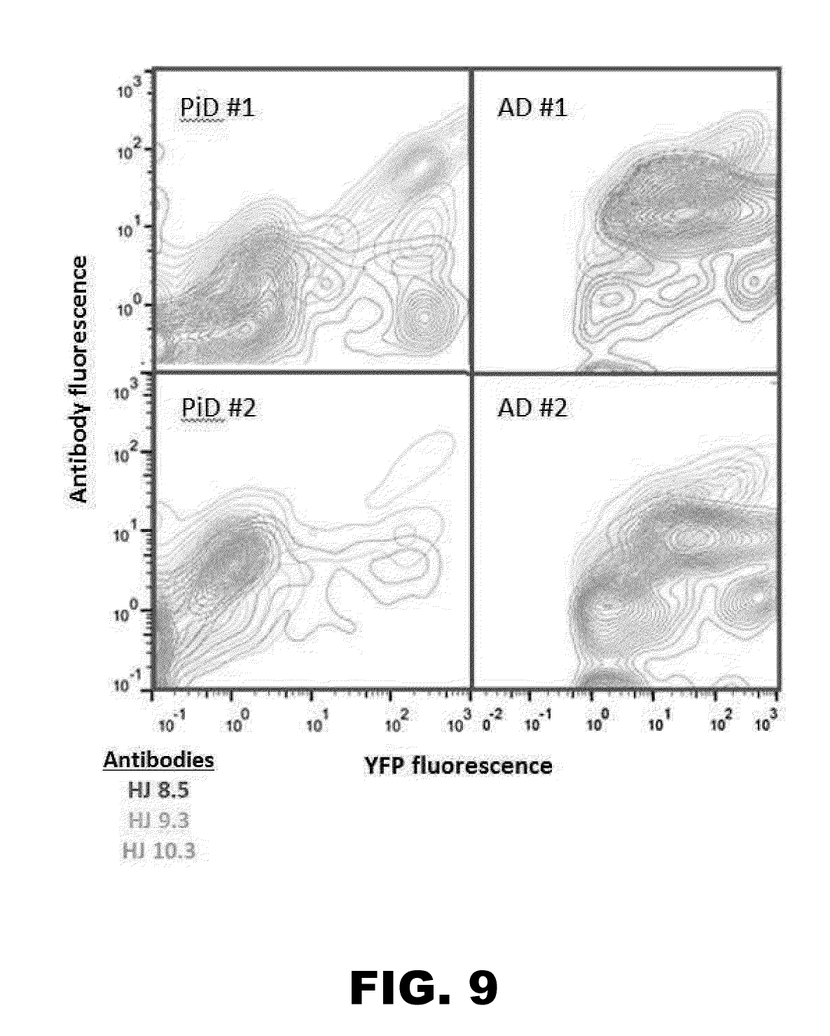

Fingerprinting cell-derived tau strains allows efficient grouping of tau conformations (FIG. 9). Brain homogenates from tauopathy patients were applied to cells producing tau RD-YFP in order to “seed” the diffuse tau and create stable cell lines able to propagate tau inclusions of various morphologies. Morphologically, two cell lines derived from a Pick's disease brain appeared to propagate the same strain. Likewise, two cell lines derived from an Alzheimer's disease brain appeared to propagate the same strain, which was morphologically distinct from the Pick's strains. These cell lines were lysed and incubated with three monoclonal antibodies. As shown, the Pick's strains display the same pattern of antibody binding and the Alzheimer's strains display the same pattern of antibody binding. These visual similarities have been confirmed with Matlab. This method is able to replicate the cellular data and group tau conformations in a more efficient and quantitative manner.

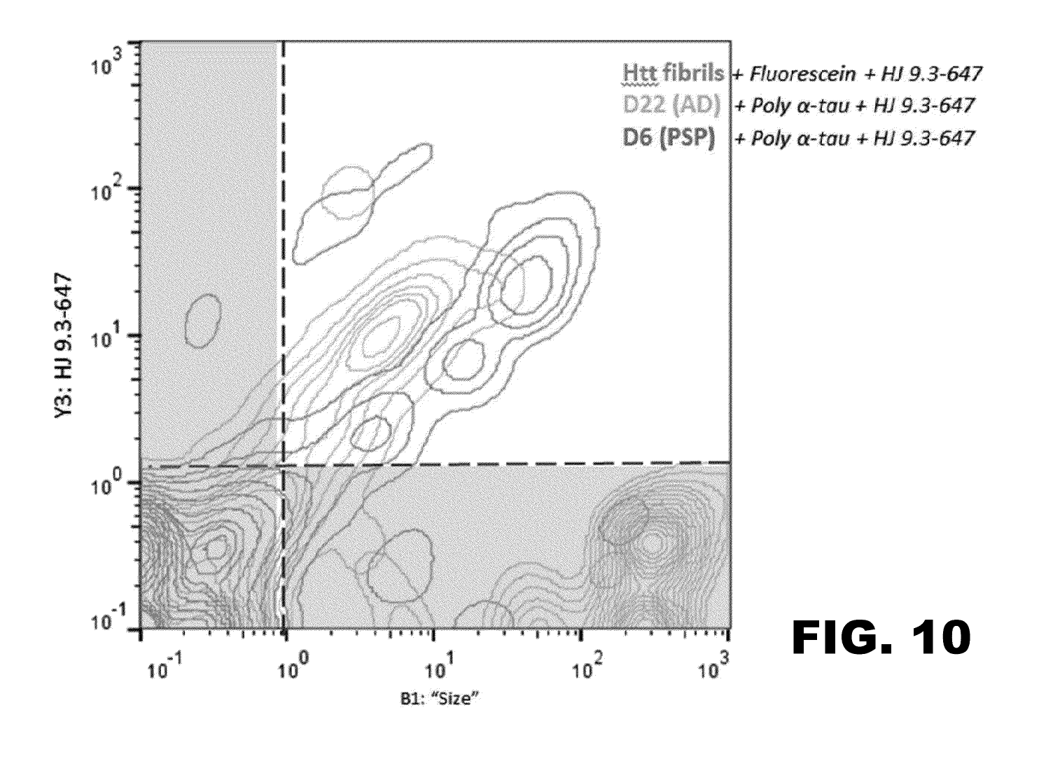

The fingerprinting method may be applied to human samples (FIG. 10). Immunoprecipitated tau from two human brains was incubated with a polyclonal anti-tau antibody and a monoclonal anti-tau antibody. The polyclonal antibody serves as a proxy for aggregate size. These samples display positive binding to the monoclonal antibody compared to Htt fibrils, used a negative control. Additionally, the samples display differential binding to the antibody, suggesting the presence of different tau strains. Once this work is extended it has the potential to identify multiple strains within a sample and group samples based on binding similarities.

### Introduction to Examples 6-10

Aggregation of the microtubule-associated protein tau underlies multiple neurodegenerative disorders collectively termed “tauopathies” (1). The tauopathies encompass myriad syndromes, including Alzheimer disease (AD), frontotemporal dementia, corticobasal degeneration (CBD), progressive supranuclear palsy (PSP), and others (1). All are relentlessly progressive, with distinct clinical and neuropathological features, but the molecular basis of this diversity is unknown. Further, because of occasional overlap in clinical and neuropathological features, standard metrics are imperfect, and there is occasionally disagreement about how precisely to classify the disorders. Prior reports have described distinct ultrastructural tau fibril characteristics in the diseases (e.g. straight filaments vs. paired helical filaments), but whether these truly represent unique structures is uncertain (2-4). Based on molecular, cellular, animal, and patient-based studies, we and others have previously proposed trans-cellular propagation of protein amyloid pathology to explain the pathogenesis of these diseases (5-10).

Prions cause neuropathology by trans-cellular spread of protein aggregates. Prions assume pathologic conformations that are self-propagating, produce predictable patterns of neuropathology, and are thus termed “strains.” By definition, strains propagate faithfully in vivo. This is based on the formation of a pathogenic “seed” that interacts with monomer to specifically template aggregate growth. Prion strains are stable over many generations in vivo, and structural analyses suggest that distinct strain structures underlie the phenotypic diversity of prion pathology (11, 12).

We originally observed that fibrillar tau stably propagates unique amyloid structures in vitro. These prion-like properties of the tau protein led us originally to propose that conformational differences in tau amyloids might underlie the phenotypic diversity of tauopathies (14). Recently we concluded that the tau protein has virtually all the biological characteristics of a bona fide prion, based on its ability to propagate distinct strains in vitro, and in vivo, and based on the identification of disease-associated strains from patient samples from 5 different tauopathy syndromes (13).

The precise description of tau strain composition in patients might ultimately underlie more accurate diagnoses and prediction of patient outcomes. However characterization of prion strains has previously been labor-intensive, time-consuming, and relatively non-quantitative, relying on protease sensitivity patterns and antibody accessibility assays (15, 16). These approaches require relatively large amounts of material for analysis, and cannot readily distinguish prionopathies based on clinical syndrome. We posited that a monoclonal antibody's affinity for an epitope within an ordered assembly might vary with the conformation of the assembly, enabling identification of aggregate conformers based on differential binding affinity. We have tested this hypothesis by developing a multiplex avidity profile (MAP) assay to monitor binding of multiple antibodies to synthetic tau fibrils of distinct conformation. We extend this method to study brain-derived tau aggregates from three different tauopathies.

### Example 6

### In Vitro Production of Tau Fibrils with Different Conformations

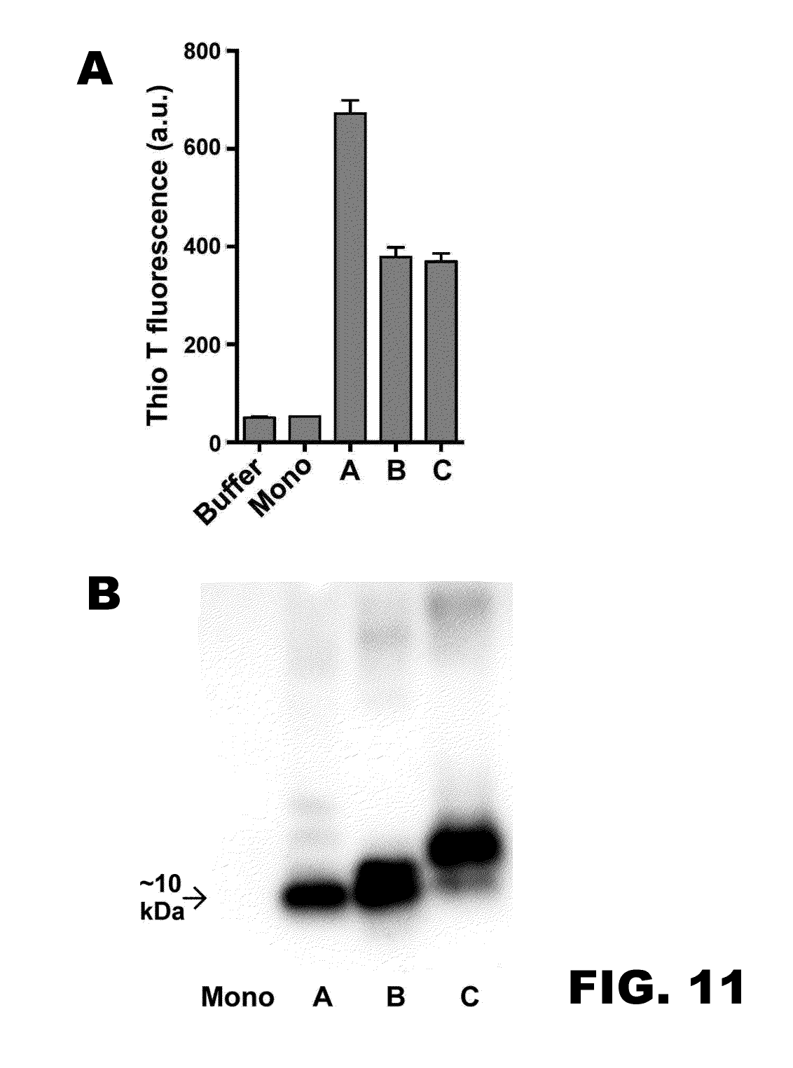

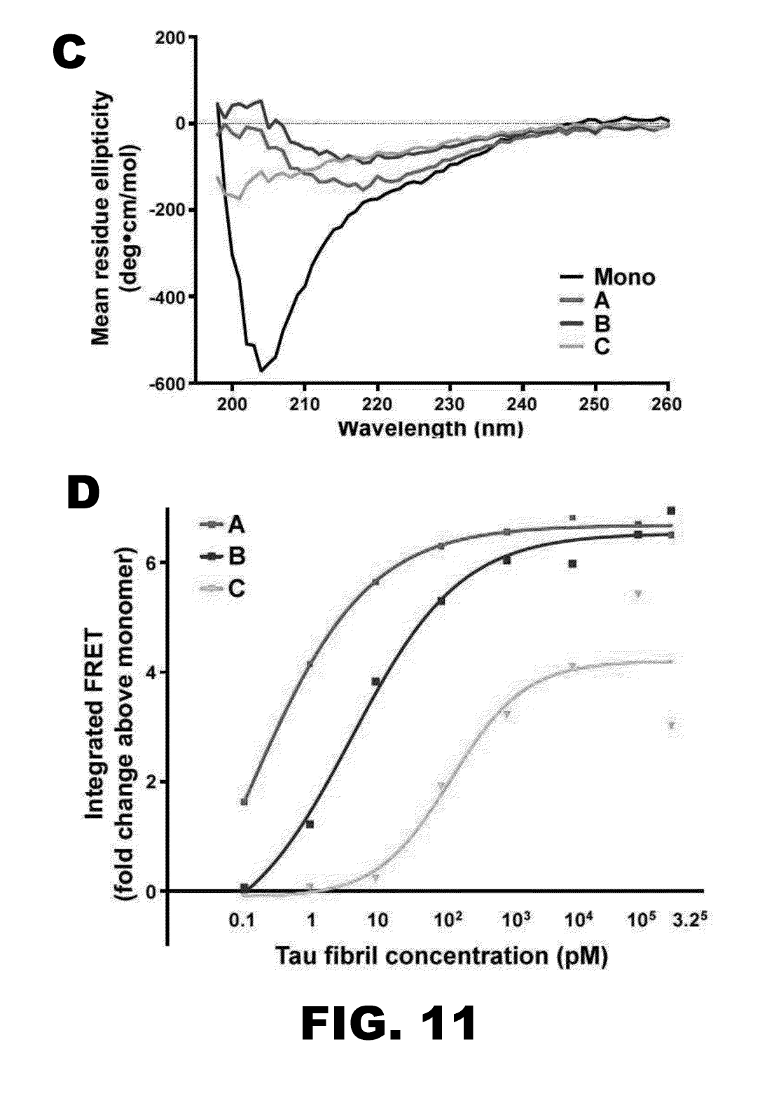

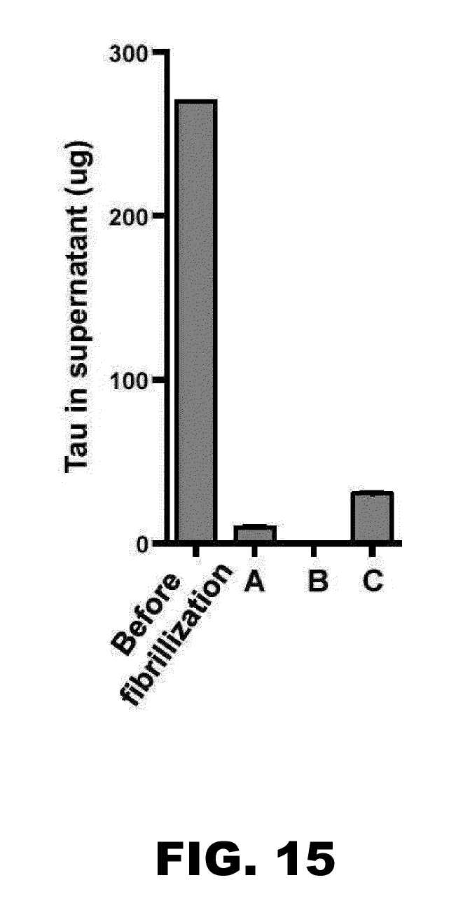

To develop the MAP assay, we first needed to create full-length tau fibrils of distinct structure, as confirmed by standard measures. Recombinant tau readily forms fibrils in vitro, and temperature and inducing agent both influence fibril conformation (17). To generate conformationally distinct aggregate populations, we fibrillized recombinant, full-length (2N,4R) monomeric tau under the three conditions (Table 1): A, with heparin (8 μM) at 37° C.; B, with heparin (8 μM) at 22° C.; C with octadecyl sulfate (50 μM) at 37° C. After 120 h, over 88% of the tau in each preparation was insoluble (FIG. 15), indicating that all reactions proceeded to near completion. We used independent studies to confirm that the fibrils had distinct structures. All fibril preparations displayed significant binding to Thioflavin T, indicating the presence of beta-sheet structures (FIG. 11A). We have previously used limited proteolysis to distinguish tau prion strains produced in cultured cells (13). We thus probed for differences in the fibril preparations using limited proteolysis with pronase, followed by western blot to detect protease-resistant fragments. We observed different patterns of protease resistant bands among the three fibril preparations. Condition A fibrils featured a single band <10 kDa, Condition B featured a doublet/heavy band ˜10 kDa, and C featured a doublet between 10 and 15 kDa (FIG. 11B). We confirmed the presence of fibrillar structures by AFM. Circular dichroism (CD) spectroscopy indicated that fibrils from conditions A and C exhibited ellipticity minima at approximately 220 nm, consistent with predominantly beta sheet structure (FIG. 11C). Fibrils from condition B exhibited primarily random coil structure, with a minimum at approximately 200 nm (FIG. 11C). Non-fibrillized tau monomer exhibited an ellipticity minimum between 200 and 205 nm, also consistent with random coil structure (FIG. 11C).

Prion strains can be discriminated based on infectious titer, or seeding activity. We have previously described a seeding assay that exploits a stable cell line expressing tau-CFP/YFP protein biosensors. Tau-CFP/YFP aggregation is measured by fluorescence resonance energy transfer (FRET) using flow cytometry (18). We used this system to test the relative seeding efficiency of each fibril preparation. All had different half maximal effective concentrations (EC₅₀s): A, <1.0 pM; B, 4.3 pM; C, 140 pM (FIG. 11D; Table 2). Importantly, we controlled for approximate aggregate load by ultracentrifuging samples after fibrillization and using the pellet fraction in all experiments. Although differences in seeding activity may be due to different size distributions of tau aggregate particles, these measures are nonetheless consistent with distinct structures of fibril preparations. Taken together with the preceding experiments, these studies indicate that the A, B, and C fibril growth conditions generated conformationally distinct fibrils.

TABLE 1
Tau fibrillization conditions.

| Sample | Inducing Agent | Temperature |
|---|---|---|
| A | 8 μM heparin | 37° C. |
| B | 8 μM heparin | 22° C. |
| C | 50 μM octadecyl Sulfate | 37° C. |

TABLE 2
Distinct fibril types display a range of seeding efficiencies.

| | A | B | C |
|---|---|---|---|
| Plateau (fold increase) | 6.67 | 6.55 | 4.19 |
| EC₅₀ (pM) | <1.0 | 4.3 | 141 |
| R² | 0.997 | 0.990 | 0.897 |

### Example 7

### Structural Discrimination by Multiplex Avidity Profile

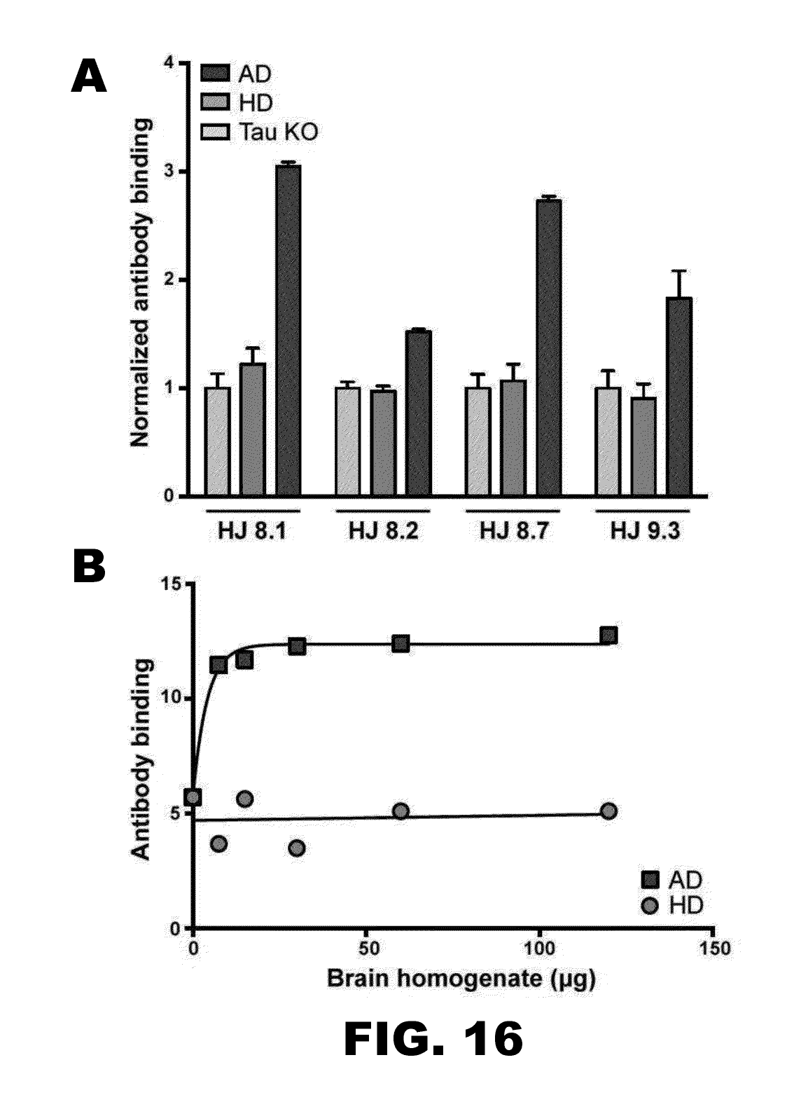

With three reference fibril preparations, we tested whether a MAP based on monoclonal antibodies would discriminate their structures. A “sandwich” format of antibody detection readily discriminates protein multimers from monomer because any one monoclonal antibody trap/detection combination must bind two epitopes to generate a signal (FIG. 12A) (19). We tested this idea by evaluating four different anti-tau monoclonal antibodies in a sandwich format, confirming that tau knockout mouse brain had no signal, human Huntington disease brain (HD) had no signal, whereas AD brain had a detectable signal across the panel, consistent with tau aggregates (FIG. 16A). This was true across a range of brain concentrations (FIG. 16B). We hypothesized that each antibody might recognize its cognate epitope with slightly different avidity, depending on the structure of the tau assembly. We anticipated that a multivariate analysis that evaluates the avidity of various antibodies for aggregates could then create a “barcode” for each structure, enabling facile discrimination of the assemblies, without the need for conformation-specific antibodies (FIG. 12B)

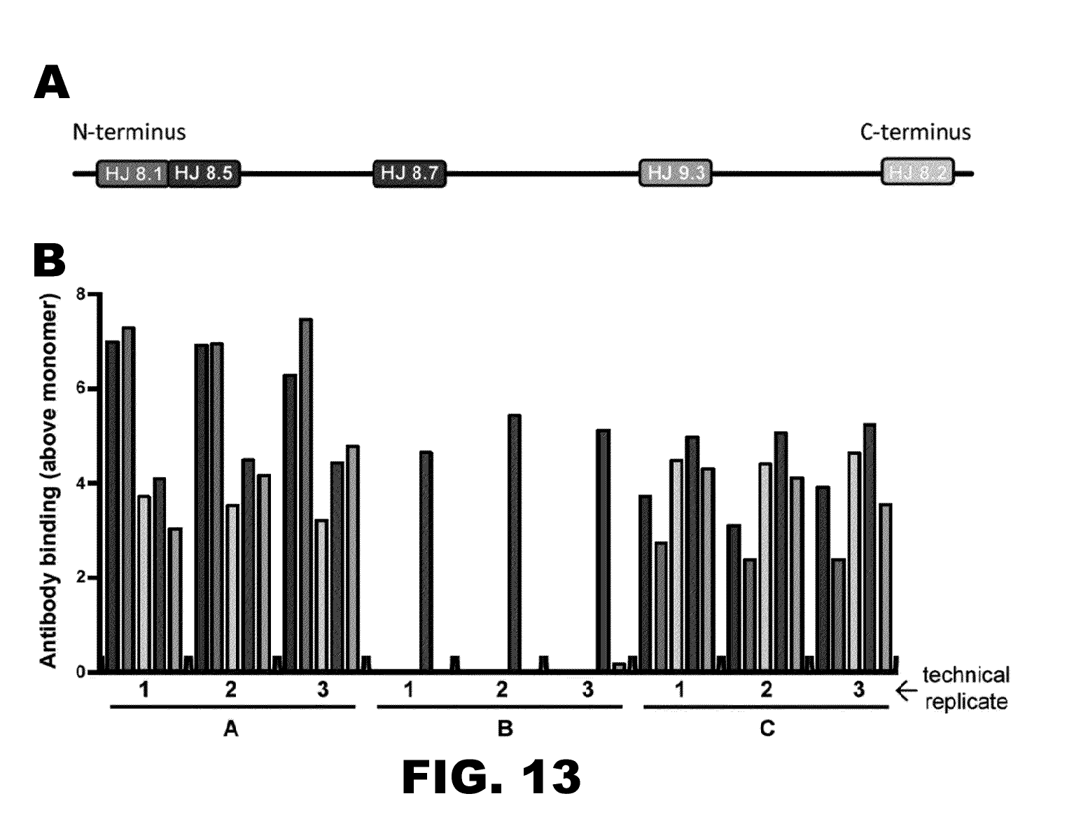

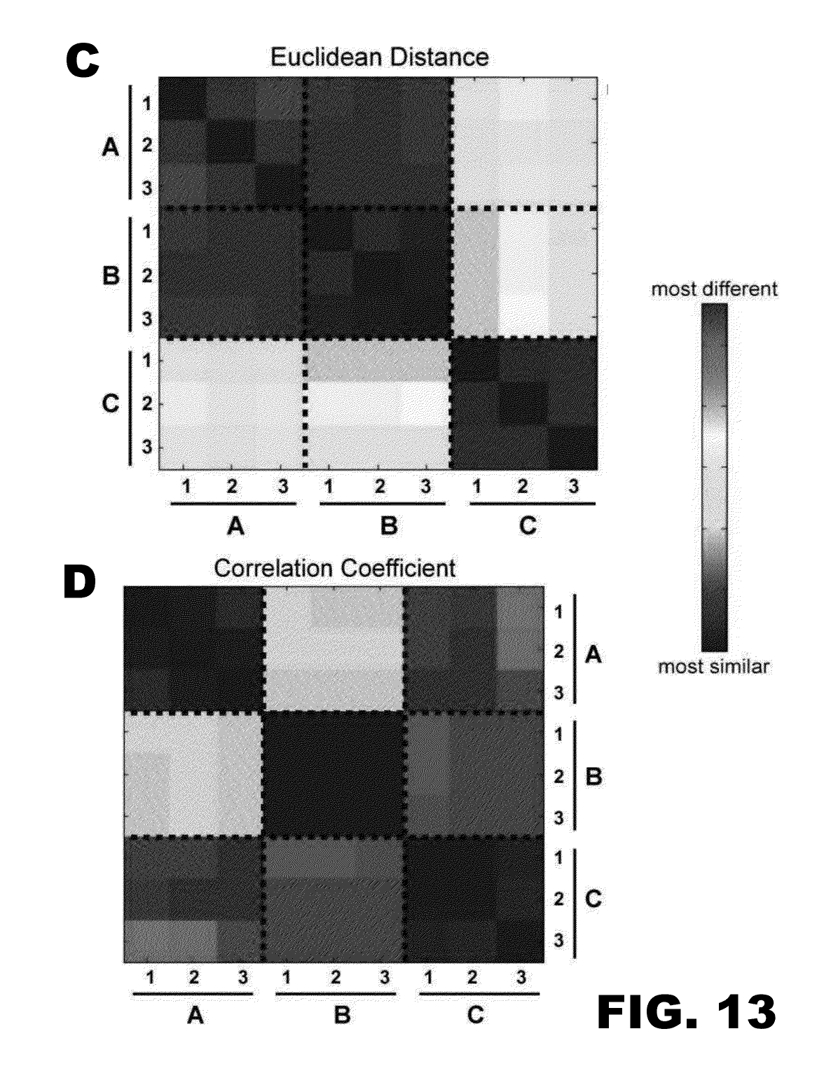

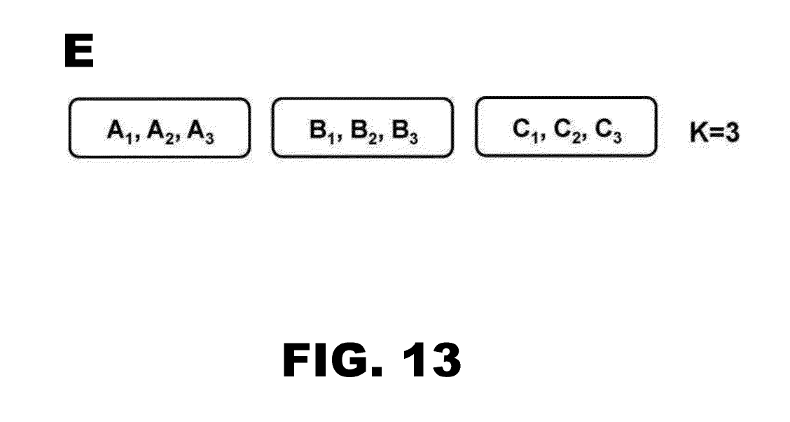

We began with five monoclonal anti-tau antibodies previously generated against full-length tau, and which recognize distinct linear epitopes within the protein (FIG. 13A; Table 3) to evaluate the three fibril preparations described in FIG. 11. Microspheres coated with each antibody were separately incubated with a saturating amount of each sample. For detection, we next incubated with the same monoclonal antibody covalently labeled with a fluorescent dye (AlexaFluor488). The five individual binding signals combined represent the MAP for each sample. Since we analyzed identical amounts of tau aggregate in each case, we initially used absolute fluorescence signals to determine the MAP.

We generated MAPs for three technical replicates of each fibril preparation (FIG. 13B), testing whether the technical replicates of a single preparation would cluster together. We plotted samples in a five-dimensional space in which each axis represents binding to an individual antibody. We predicted samples with similar binding profiles would colocalize in this space. We then calculated the Euclidean distances between all pairwise combinations of samples (FIG. 13C) and used K-means clustering to bin these samples into discrete groups. The greater the structural similarity between two samples, the smaller the Euclidean distance between them and the more likelihood they would cluster together. K-means clustering uses a computer algorithm (Matlab_R2014a) to determine in an iterative series of analyses the most optimal way to separate samples into n=K clusters. The analysis makes the assumption of K groups; if the constituents of the groups begin to vary depending on the analysis, this reflects a ‘breakdown” of the grouping, suggesting that the accurate number of truly separable groups=K−1. Using K-means clustering, the conditions A, B, and C grouped perfectly by fibril type at K=3 groups (FIG. 13E).

Whereas absolute fluorescence signal worked well to cluster recombinant fibrils, ultimately we seek to analyze patient samples in which the total amount of tau aggregates could vary. We thus used correlation coefficients to compare epitope availabilities. Rather than querying proximity in n-dimensional space, this analysis asks how the relative signal intensities of the n antibodies compare between samples. For example, if three antibodies exhibit binding signals of 2, 4, and 6 to sample A, and 20, 40, and 60 to sample B, these would not be proximal in three-dimensional space. However, their relative patterns of antibody binding are the same, and the correlation coefficient between the two samples would be 1, indicating a high degree of similarity. When analyzed by this criterion, technical replicates of each recombinant fibril type are more similar to each other than to other fibril types (FIG. 13D). Analysis by MAP thus confirmed that fibril preparations A, B, and C are structurally distinct, and that individual analyses produce stable clustering, corroborating the biochemical and biophysical differences observed by standard analyses. We anticipated that MAP might represent a facile and highly quantitative alternative to traditional methods to characterize aggregate conformation.

TABLE 3
Linear epitopes of anti-tau monoclonal antibodies

| Antibody | Linear Epitope (aa) |
|---|---|
| HJ 8.1 | 25-34 |
| HJ 8.2 | 406-412 |
| HJ 8.5 | 25-34 |
| HJ 8.7 | 118-122 |
| HJ 9.3 | 272-281 |

### Example 8

### MAP of Human Tauopathies Reveals Structural Clustering

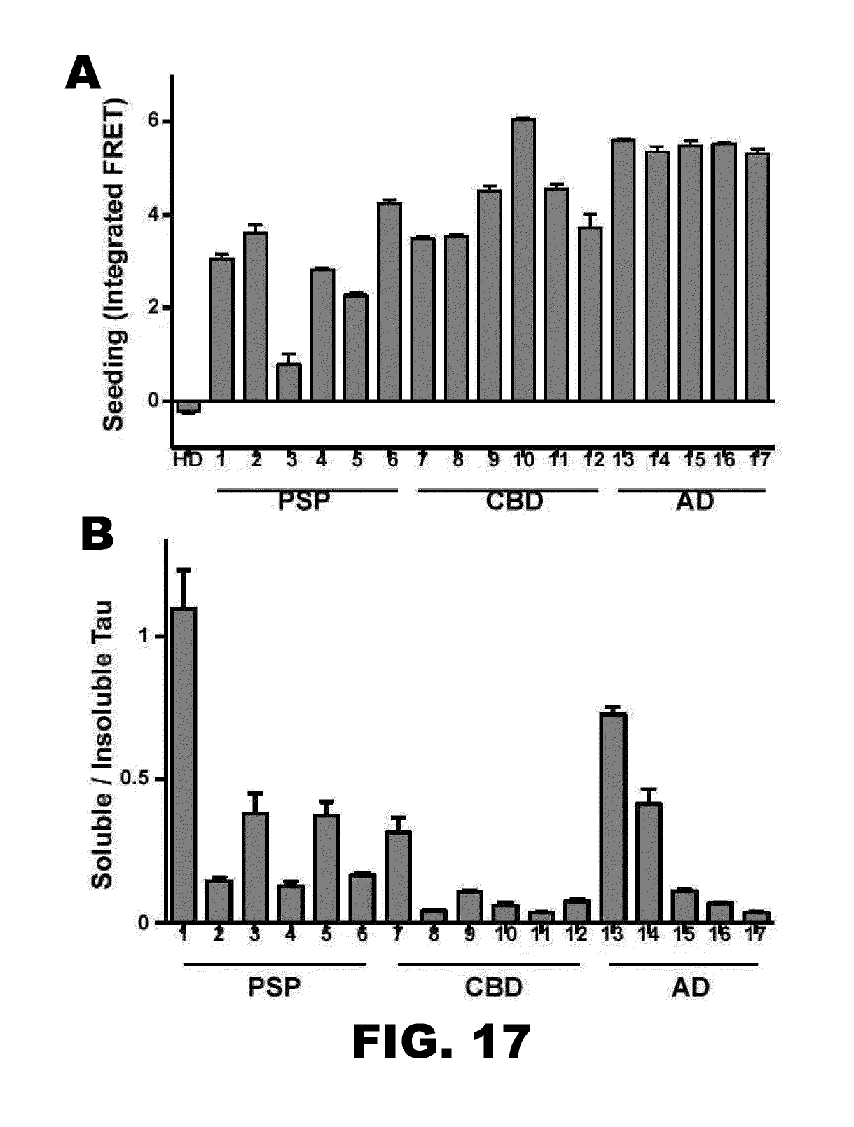

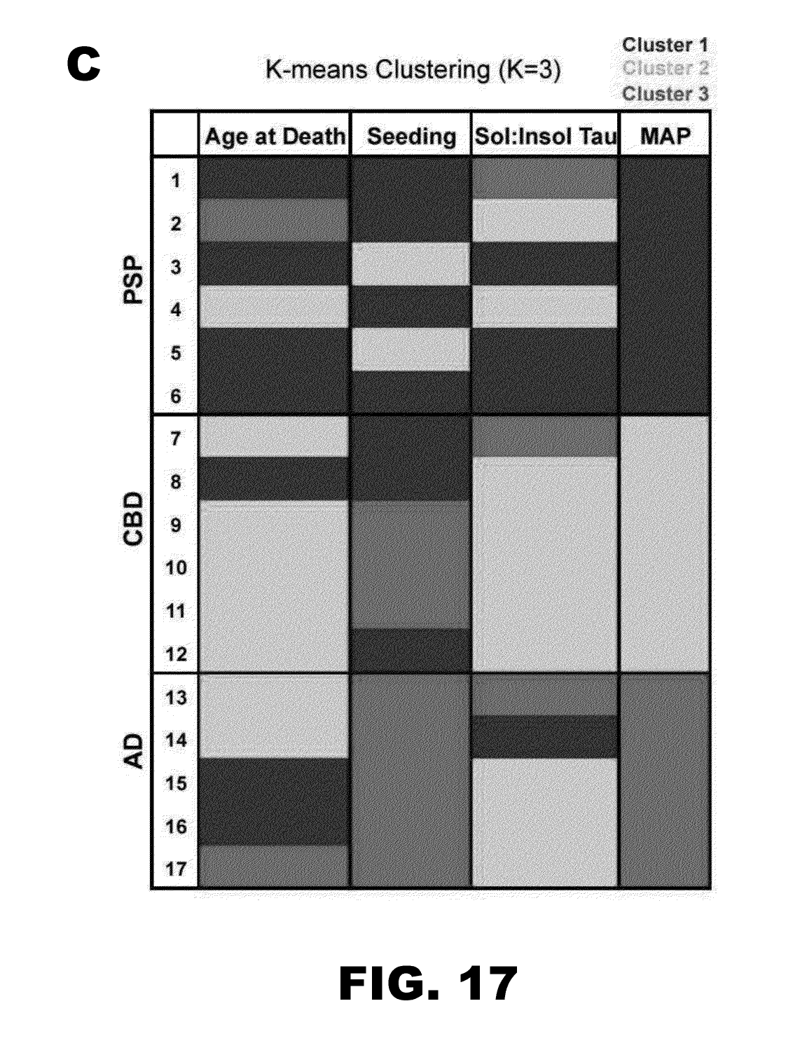

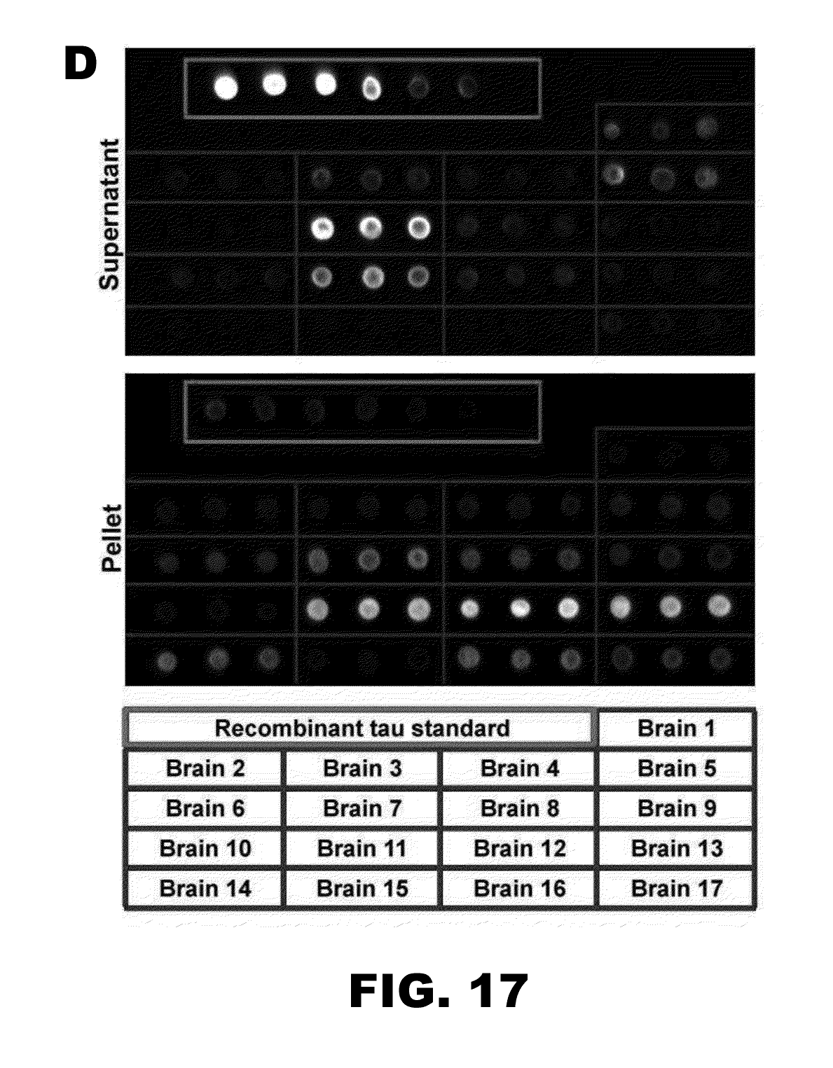

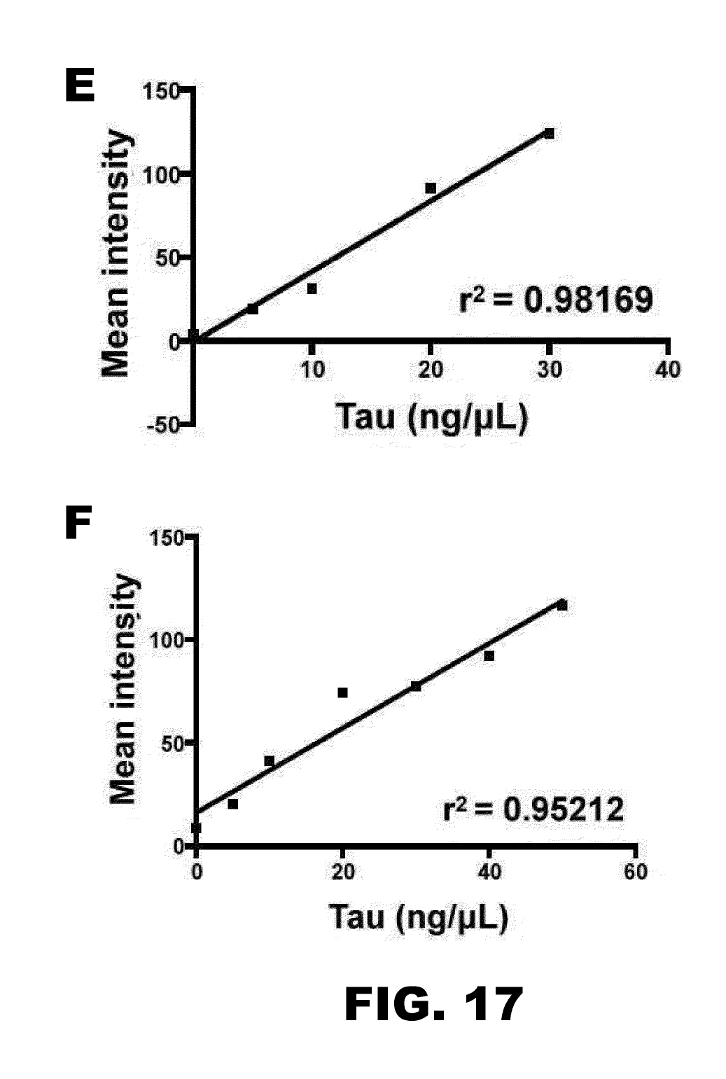

To test the utility of MAP in the analysis of human brains, we obtained brain samples dissected from the medial frontal gyrus of 17 pathologically confirmed tauopathy patients: 5 AD; 6 PSP; 6 CBD. We selected samples based on clinical syndrome in the donors, a high tau burden, and pathology considered “typical” for each syndrome. After homogenization in aqueous buffer, we determined the seeding activity of each sample using the FRET biosensor cell system (FIG. 17A), and the ratio of soluble to insoluble tau (FIG. 17B). Seeding activity alone could not group samples by tauopathy, nor could ratio of soluble to insoluble tau, or age at time of death (FIG. 17C).

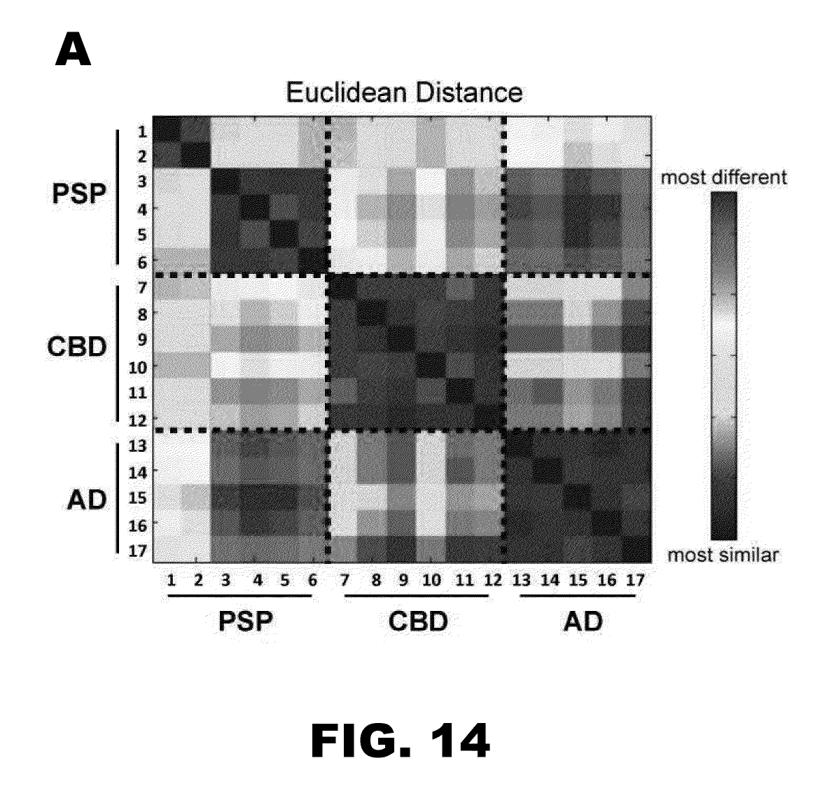

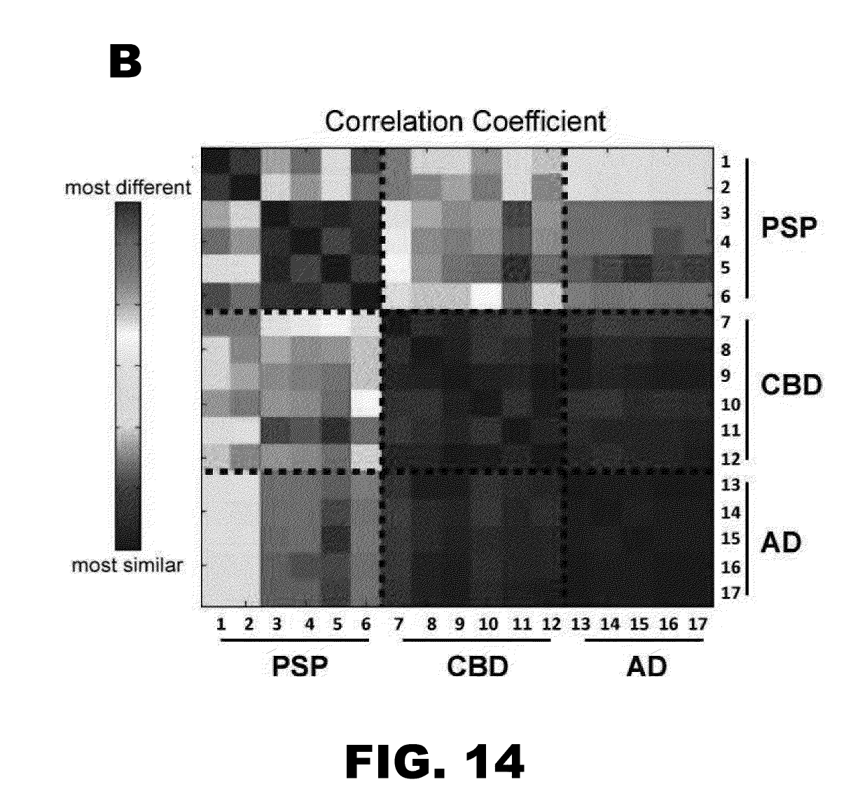

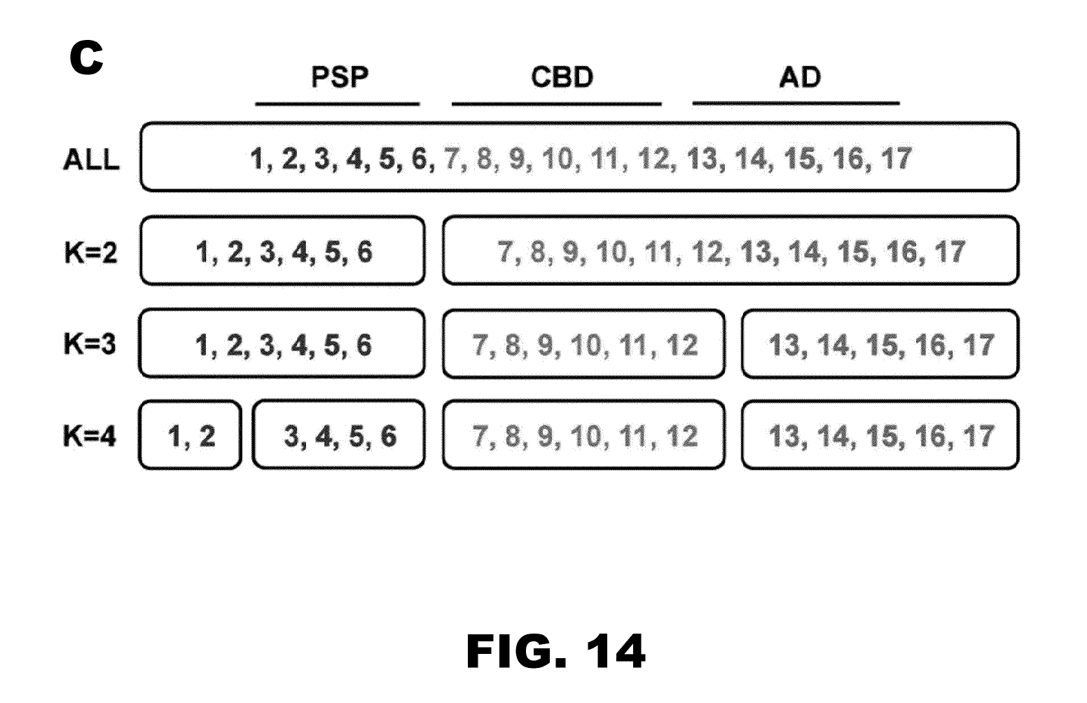

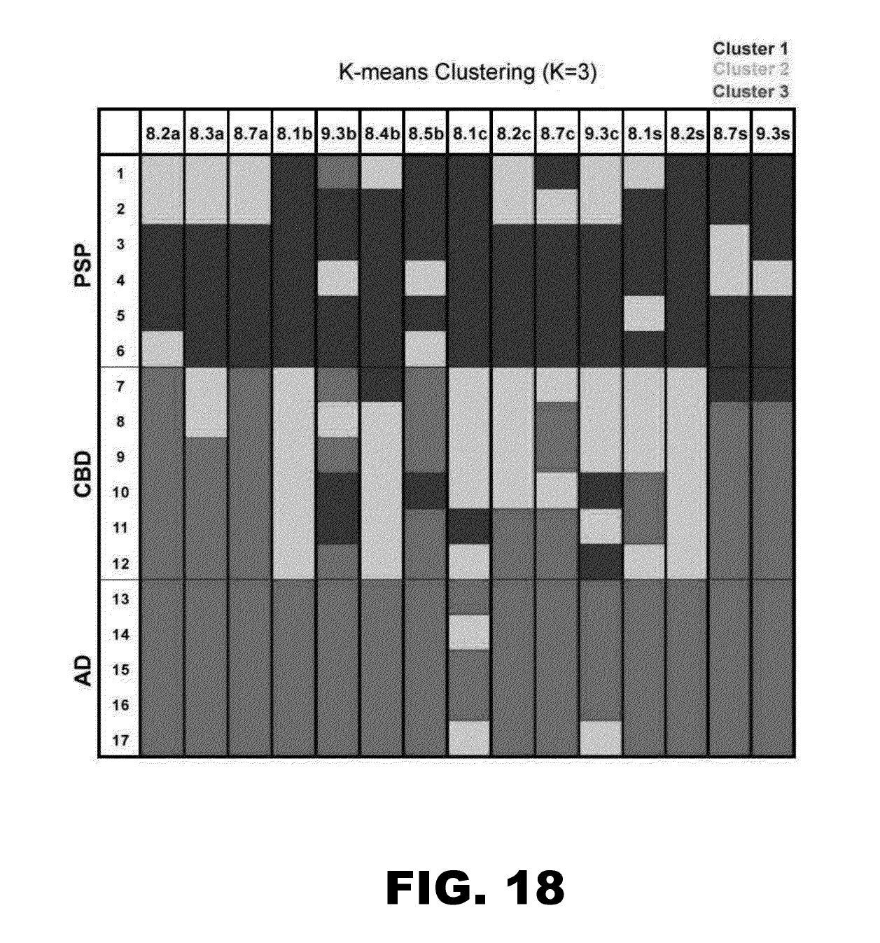

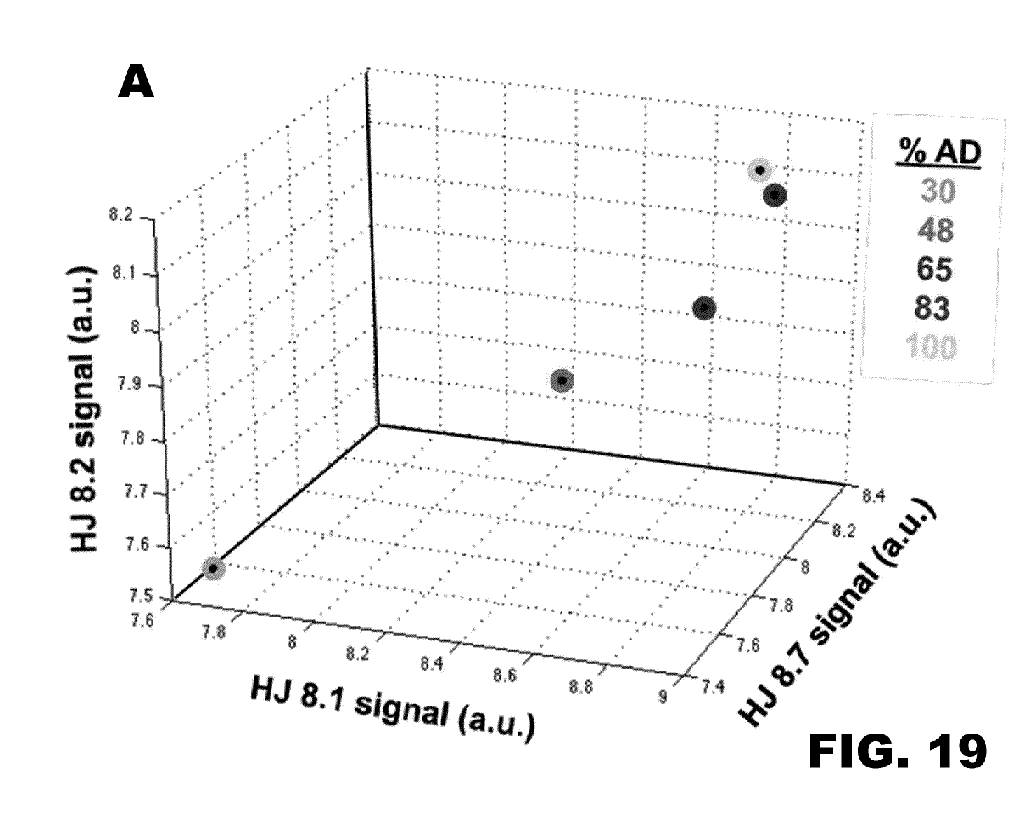

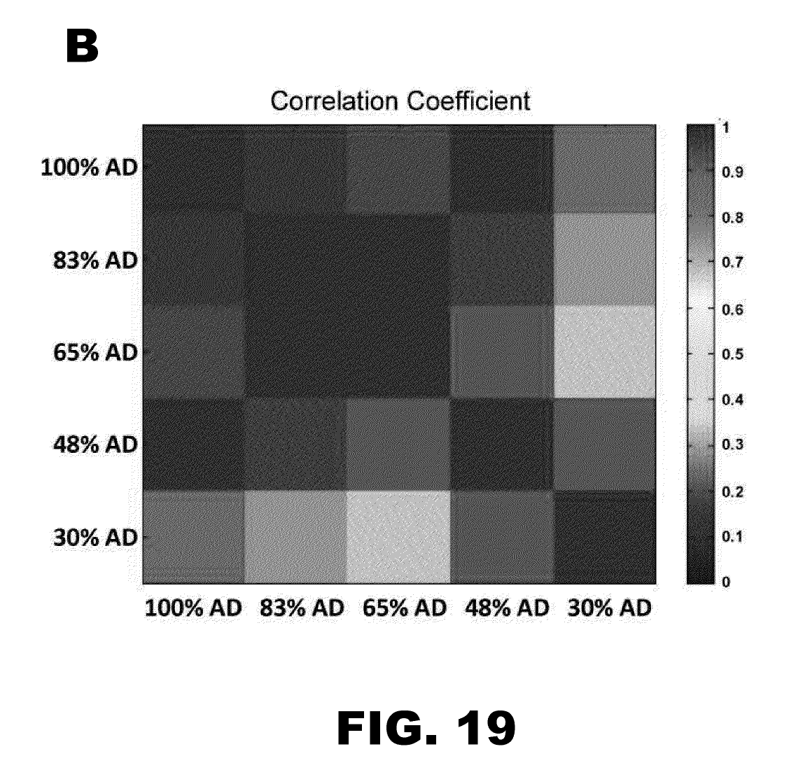

To generate MAPS for these samples, we used four monoclonal anti-antibodies (HJ 8.1, 8.2, 8.7, 9.3). We used a saturating amount of each sample (60 μg total protein) against limiting microsphere/antibody complexes. Under these conditions, we observed equivalent levels of binding to a polyclonal antibody used in the sandwich assay across samples (data not shown). We first analyzed the absolute avidities across each sample. In each case, we background-subtracted the binding signal observed from a tau KO mouse. We calculated the Euclidean distance (FIG. 14A) and correlation coefficient (FIG. 14B) for each pairwise sample combination. We used Euclidean distance to analyze the groups and compare their similarity. We observed that patients within a syndrome were most similar to one another. This was also true when we used the correlation coefficient to compare groups, although this less efficiently discriminated AD and CBD patients (FIG. 14B). When we applied cluster analysis to samples based on Euclidean distance, at K values of 2, 3, 4, and 5 (FIG. 14C). At K=3, the 17 samples clustered by tauopathy with complete specificity, indicating a high degree of similarity between patients diagnosed with the same syndrome. At K=4, two PSP samples consistently separated from the larger PSP group, suggesting that these samples are structural outliers. No individual antibody could reliably group samples by tauopathy (FIG. 18), illustrating the power of MAP. We evaluated the relationship of tau aggregate load to binding signal by testing the signal derived from AD brain with that of a patient with HD, which doesn't have tau neurofibrillary pathology. We diluted various amounts of AD brain lysate into the HD brain lysate and compared the relative antibody avidities we detected. We observed no effect of aggregate load on the correlation coefficient (i.e. relative signals among antibodies), despite reduced overall signal intensities (FIG. 19A,B). The simplest interpretation or our results is that MAP identifies disease-associated conformations for each syndrome, allowing diseases to be grouped according to the structure of their associated aggregates.

### Example 9

### Structural Categorization by MAP

Protein aggregation likely underlies pathogenesis of a variety of neurodegenerative conditions, including tauopathies, synucleinopathies, and prion diseases. Our recent work has linked tauopathy syndromes with associated tau prion strains (13). This implies that it might be possible to characterize human tauopathies by quantitatively categorizing the structure of their associated aggregates. We began by producing recombinant tau, and forming fibrils in vitro under three conditions that would produce distinct structures. We confirmed that the fibril preparations had different structures using a variety of independent methods: seeding into a biosensor cell line, CD spectroscopy, and limited proteolysis. We then developed a method to profile recombinant tau fibrils using a panel of monoclonal anti-tau antibodies. We created a bead-based “sandwich” assay that measures relative avidity of each monoclonal antibody for an aggregate. This antibody binding profile creates a “barcode” that is robust, and faithfully places each fibril preparation in multivariate space. This does not require prior knowledge of aggregate structure, or conformation-specific antibodies. In a preliminary application of this method, we then tested 17 brains of patients with distinct tauopathy syndromes, AD, CBD, PSP, in which each tissue sample was derived from the same region (inferior frontal gyrus), and had roughly the same tauopathy burden by standard histopathology. We readily grouped each syndrome based purely on aggregate profile. The chance of randomly grouping these samples in such a way is one in 7×10⁷. By contrast, all other disease related measures we tested—age at time of death, seeding activity, and ratio of soluble to insoluble tau—were insufficient to group these samples by tauopathy. These data suggest an intimate link between aggregate structure and clinical syndrome, and highlight the importance of MAP as an analytical tool.

Current methods to distinguish protein aggregate conformations in pathological specimens (e.g. differential proteolysis, electron microscopy) are mostly qualitative, making them inherently subjective and inaccurate. They are also extremely time- and labor-intensive, making impractical a large-scale analysis of human samples. These limitations have restricted our ability to quantitatively characterize the spectrum of protein aggregates that are present in human disorders, and thus to test directly the hypothesis that aggregate structure is highly correlated with tauopathy syndrome. While it is not necessary to know structure with atomic detail, it is critical to employ a metric that readily discriminates one aggregate profile from another. This is the first step towards testing whether aggregate structure predicts clinical phenotype, rate of progression, response to particular therapy, etc.

MAP uses a panel of monoclonal antibodies to determine the relative avidity of each for a tau aggregate in a sandwich detection system. This circumvents the problem of trying to develop structure-specific antibodies with binary (all or none) binding modes. This method is analogous to face recognition, which derives high accuracy by simultaneously assessing a series of characteristics, any one of which could be shared by multiple individuals. By compiling the binding signals from multiple antibodies, we generate a multiplex avidity profile (MAP) that places a given sample in multivariate space.

We initially used Euclidean distance to assess fibril structures, reasoning that the smaller the distance between two samples, the more similar they would be to one another. Analysis by K-means clustering accurately grouped the preparations. To address the potential confound of aggregate load variance in patient samples, we calculated the correlation coefficients for all pairwise sample combinations. This pairwise calculation compares the relative avidity of each monoclonal antibody vs. the others, which should be equivalent across samples containing varying aggregate loads of the same conformation. We spiked various amounts of AD brain homogenate (which contains tau aggregates) into HD brain homogenate (which contains only monomeric tau) in order to generate samples containing varying loads of the same aggregate type. We confirmed that the correlation coefficients between these samples remained high. Thus variation in aggregate load cannot account for our findings. This was also corroborated by analyzing insoluble tau via biochemistry, which did not predict syndrome clustering.

We have described an approach to MAP based on antibodies binding to aggregates. However, in theory binding of any series or combinations of small molecules, e.g. amyloid-binding agents or peptides, could be used to discriminate structures. Further, given the nature of the analysis, it is likely that multiple independent agents could even be used effectively together (for example peptide aptamers, small molecules, or antibodies). This is analogous to discrimination of different individuals based on the way a single piece of clothing, e.g. a jacket, shirt, or cape, drapes on their bodies. Finally, of course, MAP is not limited to tau, but can potentially be applied to an ordered assembly of any protein. It might be particularly useful in the analysis of peripheral amyloidoses.

### Example 10

### Linking Aggregate Structure to Syndrome

PrP^{Sc} structures are stable when propagated through mammalian hosts, and produce remarkably consistent patterns of neuropathology following inoculation. This has led to the conclusion that prion structure dictates disease, at least to a large extent. After finding that tau aggregates exhibited stable structural propagation in vitro, we initially hypothesized that distinct human tauopathies could be caused by propagation of unique tau aggregate structures (14). Our subsequent work has defined tau prion strains that stably propagate in cells and mice. Further, strains produced in cells trigger unique patterns of pathology when introduced into mice. Finally different tauopathy syndromes appear to be comprised of distinct constellations of strains that can be isolated in a clonal fashion in vitro (13). With MAP we have circumvented the time-consuming and inherently biased process of isolating and characterizing individual tau strains in cells. At described, MAP cannot describe strain complexity or diversity within an individual, since it is based on the avidity of a monoclonal antibody for the sum of all aggregates in a sample, whether comprised of a single conformer, or hundreds. However, so far, with a limited number of patient samples this integrated analysis appears sufficient to parse individuals by clinical syndrome. Future studies with larger numbers of patients will be required to determine the utility of this system, whether outliers observed by this method have any clinical or syndromic significance, and whether MAP applied to disorders due to aggregation of other proteins (Aβ, synuclein, TDP-43, etc.) will facilitate more accurate syndromic classification of their associated diseases.

### Methods for the Examples

Recombinant Tau Purification.

The wild type 2N4R tau expression plasmid (pRK172-Tau 2N4R) was a generous gift from Virginia Lee. Site directed mutagenesis to obtain 2N4R C291A/C322A was accomplished using Pfu turbo polymerase and transforming into XL 10-Gold ultracompetent cells. The mutant plasmid was transformed into BL21DE3-Gold competent cells for protein expression and induced with 1 mM IPTG at 37° C. Tau was purified as previously described (Goedert and Jakes, 1990) with the exception that to increase yield cells were lysed with a French pressure cell press rather than a probe sonicator. After cation exchange the protein was lyophilized and stored at −80° C.

Tau Fibrillization.

Lyophilized tau was resuspended in tau buffer (10 mM HEPES, 100 mM NaCl) and ultracentrifuged at 130,000×g for 1.5 hours in order to eliminate any preexisting high molecular weight species. The supernatant fraction was fibrillized under the conditions listed in Table 1. After 120 hours of undisrupted fibrillization the fibril preparations were ultracentrifuged at 130,000×g for 1.5 hours. Protein concentration in the supernatant fraction was measured using Bradford reagent and subtracted from the initial concentration in order to obtain an accurate measure of protein in the pellet fraction. The pellet fraction was then resuspended to 2 uM tau in tau buffer and sonicated for 90 minutes using a water bath sonicator. After sonication the fibrils were snap frozen and stored at −80° C. in single-use aliquots.

Thioflavin T Assay.

After sonication each sample was incubated with 40 μM ThioflavinT to a final concentration of 1 μM tau. Fluorescence signal was recorded with 440 nm (+/−5 nm) excitation and 480 nm (+/−5 nm) emission using a Tecan 1000 plate reader.

Limited Proteolysis.

Pronase (Roche) was diluted in PBS to a final concentration of 1 mg/mL and stored at −80° C. in single-use aliquots. 1 ug of each sample was added to 10 μL of pronase (at 25 μg/mL) and raised to a final volume of 20 μL. After a 1-hour incubation at 37° C. the reaction vas quenched with 20 μL of 2× sample buffer to a final concentration of 1% SDS. The quenched solution was placed in a heat block at 95° C. for 5 minutes. 10 μl of each sample was run on a 10% Bis-Tris NuPAGE gel (Novex by Life Technologies) at 150V for 65 minutes. The protein was transferred to Immobilin P (Millipore) at 20V for 1 hour using a Semi-dry transfer apparatus (Biorad).

The membrane was blocked for 1 hour with 5% milk then probed with a rabbit polyclonal anti-tau antibody (ab64193, Abcam) using a 1:2000 dilution. After an overnight incubation at 4° C. the membrane was washed four times with 0.05% TBS-Tween and counter-probed with a goat anti-rabbit HRP antibody (Jackson Immunotherapy) at a 1:4000 dilution for 1.5 hours. The membrane was again washed four times with 0.05% TBS-Tween and once with TBS. Finally, the membrane was imaged by exposure to ECL Prime Western Blotting Detection Reagent (Fisher Scientific) for 2 minutes and developed using a digital Syngene imager.

Circular Dichroism.

Far-UV circular dichroism (CD) measurements were performed at 25° C. on a Jasco J-810 spectropolaimeter using a 0.1 cm optical path length. 200 μL of each sample (at 2 uM tau) were scanned continuously from 198 nm to 260 nm. The reported spectra are the average of 20 scans with a data pitch of 1 nm.

Cell Culture.

HEK293 cells were grown in Dulbecco's modified Eagle's medium (Gibco) augmented with 10% fetal bovine serum (HyClone) and 1% penicillin/streptomycin (Gibco). Cells were maintained at 37° C. and 5% CO2 in a humidified incubator.

FRET Flow Cytometry.

FRET biosensor cell lines described previously (Holmes and Furman, 2014) were plated at a density of 50,000 cells/well in a 96-well plate. Eight hours later, at 50% cell density, samples were transduced into cells using Lipofectamine 2000. After a 48-hour incubation at 37° C. cells were harvested with 0.05% trypsin and fixed in 2% paraformaldehyde (Electron Microscopy Services) for 15 min, then resuspended in PBS. The MACSQuant VYB (Miltenyi) was used to perform FRET flow cytometry as previously described (ibid). FRET quantification was accomplished via a visually guided gating strategy using FlowJo v10 software (Treestar Inc.). Ultimately, the integrated FRET density (derived by multiplying the percent of FRET-positive cells in each sample by the median FRET intensity of those cells) was compared across samples.

Antibody Labeling.

AlexaFluor 488 was reconstituted in distilled H₂O and aliquotted into 25 ug aliquots for single use. These were vacuufuged for 15 mins (in order to eliminate water) and stored at −20° C. 35 ug of each Ab was added to 25 ug of the dye in 100 uL of PBS. These were incubated overnight at 4° C. and then dialyzed to remove any unconjugated free dye.

Multiplex Avidity Profiling.

2.5×10⁶ streptavidin-coated microspheres (Bangs Laboratory; 10 um diameter) were incubated with 10 ug of each biotinylated AbM for 2 hours at room temperature and subsequently centrifuged at 5000×g for 3 minutes and resuspended in PBS. All incubations were conducted in 1.5 mL tubes with top-over-bottom rotation. 60,000 microsphere-antibody complexes were incubated with each sample (1 ug of recombinant protein or 60 ug of total brain homogenate) in a final volume of 300 uL MAP buffer (% BSA in PBS). After an overnight incubation at 4° C., samples were centrifuged at 5000×g for 3 minutes, resuspended in 300 uL of MAP buffer, and incubated with the respective fluorescently labeled AbM for 6 hours at room temperature. Samples were subsequently centrifuged at 5000×g for 3 minutes and resuspended in 200 uL of PBS. The MACSQuant VYB (Miltenyi) was used to perform flow cytometry. Forward scatter area and height, side scatter area and height, and either AF647 or AF488 fluorescence (depending on the dye used to label the antibodies) were measured. AF647 was stimulated with the 647 nm laser and fluorescence was captured with a nm filter, while AF488 was stimulated with the 488 nm laser and fluorescence was captured with a 525/50 nm filter.

Flow Cytometry Analysis:

FlowJo v10 software (Treestar Inc.) was used to analyze all flow cytometry data. Events were initially gated for size in order to exclude sub- or multi-microsphere particles. A sample containing no antigen was used to create an arbitrary threshold of fluorescence positivity for each AbM: between 0.5 and 1% of the microspheres in the “no antigen” condition were deemed positive. This gate was applied to all samples of a particular AbM. “Integrated fluorescence” (IF) was calculated for each sample-antibody combination by multiplying the percent of fluorescent-positive microspheres by the median fluorescence intensity of those microspheres. Finally, the natural log was calculated for each measure of IF.

MAP Analysis.

Logarithmically transformed values of IF were used for all subsequent calculations. The Euclidean distance between two samples, X and Y, was calculated using the formula: √[(AbM_{1X}−AbM_{1Y})²+(AbM_{2S}−AbM_{2Y})²+ . . . +(AbM_{nX}−AbM_{nY})²], in which AbMs 1 through n represent the individual antibodies used. Correlation coefficients between samples were calculated using Graphpad Prism 6 software. A matrix comparing binding signals across all samples and antibodies was uploaded to Matlab_R2014 for K means clustering analysis. 10,000 iterations were conducted at each K value in order to determine the most stable clusters.

### REFERENCES FOR THE EXAMPLES

1. Lee, V. M., Goedert, M., and Trojanowski, J. Q. (2001). Neurodegenerative tauopathies. Annu. Rev. Neurosci. 24, 1121-1159.
2. Crowther, R. A. and Goedert, M. (2000). Abnormal tau-containing filaments in neurodegenerative diseases. J Struct Biol 130, 271-279.
3. Tomonaga, M. (1977). Ultrastructure of neurofibrillary tangles in progressive supranuclear palsy. Acta Neuropathol. 37, 177-181.
4. Buee, L., Bussiere, T., Buee-Scherrer, V., Delacourte, A., and Hof, P. R. (2000). Tau protein isoforms, phosphorylation and role in neurodegenerative disorders. Brain Res. 33, 95-130.
5. Frost, B., Jacks, R. L., and Diamond, M. I. (2009). Propagation of tau misfolding from the outside to the inside of a cell. J. Biol. Chem. 284, 12845-12852.
6. Frost, B., and Diamond, M. I. (2010). Prion-like mechanisms in neurodegenerative diseases. Nat. Rev. Neurosci. 11, 155-159.
7. Clavaguera, F., Lavenir, I., Falcon, B., Frank, S., Goedert, M., and Tolnay, M. (2013b). “Prion-like” templated misfolding in tauopathies. Brain Pathol. 23, 342-349.
8. Calignon, A., Polydoro, M., Suarez-Calvet, M., William, C., Adamowicz, D. H., Kopeikina, K. J., Pitstick, R., Sahara, N., Ashe, K. H., Carlson, G. H., Spires-Jones, T. L., and Hyman, B. T. (2012). Propagation of Tau Pathology in a Model of Early Alzheimer's Disease. Neuron 73, 685-697.
9. Liu, L., Drouet, V., Wu, J. W., Witter, M. P., Small, S. A., Clelland, C., and Duff, K. (2012). Trans-synaptic spread of tau pathology in vivo. PLoS ONE 7, e31302.
10. Seeley, W. W., Crawford, R. K., Zhou, J., Miller, B. L., and Greicius, M. D. (2009). Neurodegenerative diseases target large-scale human brain networks. Neuron 62, 42-52.
11. Legname, G., Nguyen, H.-O. B., Peretz, D., Cohen, F. E., DeArmond, S. J., and Prusiner, S. B. (2006). Continuum of prion protein structures enciphers a multitude of prion isolate-specified phenotypes. Proc. Natl. Acad. Sci. USA 103 19105-19110.
12. Collinge, J., and Clarke, A. R. (2007). A general model of prion strains and their pathogenicity. Science 318, 930-936.
13. Sanders, D. W., Kaufman, S. K., DeVos, S. L., Sharma, A. M., Mirbaha, H., Li, A., Barker, S. J., Foley, A. C., Thorpe, J. R., Serpell, L. C., Miller, T. M., Grinberg, L. T., Seeley, W. W., and Diamond, M. I. (2014). Distinct tau prion strains propogate in cells and mice and define different tauopathies. Neuron 82, 1271-1288.
14. Frost, B., Ollesch, J., Wille, H., and Diamond, M. I. (2009). Conformational diversity of wild-type Tau fibrils specified by templated conformation change. J. Biol. Chem. 284, 3546-3551.
15. Safar, J., Wille, H., Itri, V., Groth, D., Serban, H., Torchia, M., Cohen, F. E., and Prusiner, S. B. (1998). Eight prion strains have PrPsc molecules with different conformations. Nat. Medicine 4, 1157-1165.
16. Scott, M. R., Groth, D., Tatzelt, J., Torchia, M., Tremblay, P., DeArmond, S. J., and Prusiner, S. B. (1997). Propagation of Prion Strains through Specific Conformers of the Prion Protein. Journal of Virology 71, 9032-9044.
17. Stine, W. B., Dahlgren, K. N., Krafft, G. A., and LaDu, M. J. (2003). In Vitro Characterization of Conditions for Amyloid-β Peptide Oligomerization and Fibrillogenesis. J. Biol. Chem. 278, 11612-11622.
18. Holmes, B. H., Furman, J. L., Mahan, T. E., Yamasaki, T. R., Eades, W. C., Belaygorod, L., Cairns, N. J., Holtzman, D. M., and Diamond, M. I. (2014, in press). Proteopathic Tau Seeding Precedes Histopathological Abnormalities in a Model of Tauopathy. PNAS.
19. Esparza, T. J., Zhao, H., Cirrito, J. R., Cairns, N. J., Bateman, R. J., Holtzman, D. M., and Brody, D. L. (2013). Amyloid-beta oligomerization in Alzheimer dementia versus high-pathology controls. Annals of Neurol. 73, 104-119.
20. Goedert, M. and Jakes, R. (1990). Expression of separate isoforms of human tau protein: correlation with the tau pattern in brain and effects on tubulin polymerization. EMBO J. 9, 4225-4230.

What is claimed is:

1. A method for classifying a protein aggregate, the method comprising
(a) contacting the protein aggregate with x number of epitope-binding agents, wherein x≧3 and none of the epitope-binding agents bind the same epitope;
(b) contacting the product of step (a) with y number of a epitope-binding agents linked to a label (“labeled epitope-binding agents”) to form y types of labeled aggregate, wherein y=x and the labeled epitope-binding agents of step (b) and the epitope-binding agents from step (a) collectively recognize the same epitopes;
(c) measuring the amount of label for each type of labeled aggregate, wherein the amount of the label is directly proportional to binding avidity of the labeled epitope-binding agent for the protein aggregate; and
(d) classifying the protein aggregate by assigning the protein aggregate to a discrete spatial location within a multivariate space having n number of axes, wherein n=y and each axis corresponds to a labeled epitope-binding agent, and wherein a coordinate along the axis is the binding avidity as measured in step (c).

2. The method of claim 1, wherein x≧5.

3. The method of claim 1, wherein the epitope-binding agents from step (a) and the epitope-binding agents from step (b) are independently selected from the group consisting of antibody and aptamer.

4. The method of claim 3, wherein the epitope-binding agents from step (a) and the epitope-binding agents from step (b) are antibodies.

5. The method of claim 3, wherein the antibody is selected from the group consisting of a monoclonal antibody, an Fab fragment, and an aptamer.

6. The method of claim 1, wherein the epitope-binding agents of step (a) and epitope-binding agents of step (b) are the same, with the provisio that the epitope-binding agents from step (b) are linked to a label.

7. The method of claim 1, wherein the protein aggregate is an amyloid.

8. The method of claim 1, wherein the protein is selected from the group consisting of prion protein, tau protein, alpha-synuclein protein, amyloid beta peptide, TDP-43, and htt.

9. The method of claim 1, wherein the epitope-binding agents of step (a) are linked to a solid surface, and the label is a fluorescent compound.

10. The method of claim 1, wherein the protein aggregate is in a sample selected from the group consisting of brain tissue, spinal cord tissue, cerebrospinal fluid, interstitial fluid, and blood.

11. The method of claim 9, the method further comprising isolating the protein aggregate from the sample prior to step (a).

12. The method of claim 9, wherein the sample is obtained from a subject diagnosed with a neurodegenerative disease associated with the pathological aggregation or a subject with clinical signs or symptoms of a neurodegenerative disease associated with the pathological protein aggregation.

13. The method of claim 11, the method further comprising assigning the spatial location of the protein aggregate within the multivariate space to the neurodegenerative disease of the subject.

14. A method for comparing the similarity of two or more protein aggregates, the method comprising:
(a) classifying each protein aggregate, wherein the method of classifying comprises:
(1) contacting the protein aggregate with x number of epitope-binding agents, wherein x≧3 and none of the epitope-binding agents bind the same epitope;
(2) contacting the product of step (1) with y number of a epitope-binding agents linked to a label (“labeled epitope-binding agents”) to form y types of labeled aggregates, wherein y=x and the labeled epitope-binding agents of step (2) and the epitope-binding agents from step (1) collectively recognize the same epitopes;
(3) measuring the amount of label for type of labeled aggregate, wherein the amount of the label is directly proportional to binding avidity of the labeled epitope-binding agent for the protein aggregate; and
(4) classifying the protein aggregate by assigning the protein aggregate to a discrete spatial location within a multivariate space having n number of axes, wherein n=y and each axis corresponds to a labeled epitope-binding agent, and wherein a coordinate along the axis is the binding avidity as measured in step (3).
(b) calculating a degree of similarity between the two or more protein aggregates.

15. The method of claim 14, wherein the degree of similarity is calculated by determining a Euclidean distance between the spatial locations or a correlation coefficient between the binding avidities of each aggregate.

16. The method of claim 13, wherein x≧5.

17. The method of claim 13, wherein the epitope-binding agents from step (a) and the epitope-binding agents from step (b) are independently selected from the group consisting of antibody and aptamer.

18. The method of claim 13, wherein the protein aggregate is an amyloid.

19. The method of claim 13, wherein the epitope-binding agents of step (a) are linked to a solid surface, and the label is a fluorescent compound.

20. The method of claim 13, wherein the protein aggregate is in a sample selected from the group consisting of brain tissue, spinal cord tissue, cerebrospinal fluid, interstitial fluid, and blood.
